(19) United States
(12) Patent Application Publication
(10) Pub. No.: US 2024/0377328 A1
(43) Pub. Date: Nov. 14, 2024

(54) Determining Properties of Samples Using Quantum Sensing

(71) Applicants: The University of Melbourne, Melbourne, Victoria (AU); The Florey Institute of Neuroscience & Mental Health, Parkville, Victoria (AU)

(72) Inventors: Gawain MCCOLL, Parkville, Victoria (AU); David Allan SIMPSON, Melbourne, Victoria (AU); Liam Terres HALL, Melbourne, Victoria (AU); Erin Susanne GRANT, Melbourne, Victoria (AU)

(21) Appl. No.: 18/686,805

(22) PCT Filed: Aug. 24, 2022
(86) PCT No.: PCT/AU2022/050983

§ 371 Date: Feb. 26, 2024

(30) Foreign Application Priority Data
Aug. 27, 2021 (AU) 2021902794
May 4, 2022 (AU) 2022901174

(51) Int. Cl. G01N 21/64; G01N 33/90

(52) U.S. Cl. CPC G01N 21/6489; G01N 21/6408; G01N 21/6486; G01N 33/90; G01N 2201/127

(57) ABSTRACT

A method for determining one or more properties of a molecular metal ligand in a sample comprises the steps of: providing a quantum sensor: exposing the quantum sensor to the sample: applying an illumination signal to the quantum sensor for a first predetermined duration: and detecting a photoluminescence intensity emitted from the quantum sensor. A characteristic of the detected photoluminescence intensity is indicative of one of the properties of the molecular metal ligand in the sample.

This application claims priority from Australian Provisional Patent Application No. 2021902794 filed on 27 Aug. 2021 and Australian Provisional Patent Application No. 2022901174 filed on 4 May 2022, the contents of both of which are to be taken as incorporated herein by this reference.

### TECHNICAL FIELD

The present disclosure relates to use of a quantum sensor to determine metal loading of a molecular metal ligand or metal binding agent. It relates specifically, but not exclusively to use of a quantum sensor in the form of a defect in a semiconductor material (such as a NV defect in diamond) to detect iron loading of a molecule.

### BACKGROUND OF INVENTION

It has been estimated by the World Health Organisation that at least one third of the world's population suffer from iron deficiency. The current gold standard for the clinical diagnosis of iron deficiency is a direct test via a bone marrow biopsy and iron staining. However bone marrow aspiration is far too invasive for routine use. Consequently, indirect enzyme linked immunoassays (ELISAs) are generally used on traditional blood samples where iron deficiency is suspected.

Blood bank services currently screen for anaemia by testing haemoglobin levels. However, the body preferentially loads haemoglobin with iron such that an individual may have a body iron storage deficiency (known as iron deficiency anaemia) that is not detected with a haemoglobin test. This is problematic for blood donors with undetected iron deficiency anaemia as they will become anaemic as a consequence of donating blood. Conversely, iron overload increases susceptibility to infectious diseases and excess iron stored in major organs can lead to significant health problems.

Ferritin is the body's primary iron storage protein and is found in most cells and numerous extracellular fluids of the body. It is recognised as a critical, robust and routinely used blood based biomarker for assessing iron load. The iron load in ferritin can vary widely, from empty up to the theoretical capacity of 4,500 iron atoms per ferritin. Throughout this document, all references to ‘ferritin’ should be taken to refer to the fully formed 24 subunit protein itself. When discussing the iron that is bound to ferritin, this will be referenced explicitly. In healthy people, serum ferritin levels correlate with total iron load however, under specific diseases or conditions, this relationship is confounded.

For example, ferritin levels increase in response to inflammation and chronic disease (e.g. diabetes, obesity, renal failure, cardiovascular diseases, alcoholism, various auto-immune disorders and cancer). Other common conditions that cause elevated serum ferritin include metabolic syndrome and fatty liver disease, which affects 40% of Australians over the age of 50. These conditions make iron deficiency or overload difficult to diagnose.

Alternate methods for assessing body iron storage are either invasive (e.g. liver biopsy) or expensive (MRI). FerriScan is an MRI-based method of measuring liver iron concentration. Although it provides an accurate, accessible and safe method of assessing the presence and severity of iron overload, FerriScan is cost prohibitive to many. Consequently, blood tests evaluating serum ferritin levels are primarily used in the diagnosis and management of iron overload, despite these measures of iron load being recognised as suboptimal and problematic.

The level of iron in serum ferritin is reported to correlate well with total body iron stores, however current methods to determine ferritin bound iron are research methods only (e.g. species-specific isotope dilution mass spectrometry) that cannot translate to a clinical test.

It would be desirable to overcome or at least ameliorate at least one of the aforementioned problems.

The discussion of the background to the invention included herein including reference to documents, acts, materials, devices, articles and the like is included to explain the context of the present invention. This is not to be taken as an admission or a suggestion that any of the material referred to was published, known or part of the common general knowledge as at the priority date of any of the claims.

### SUMMARY OF INVENTION

Viewed from one aspect, the present disclosure provides a method for determining one or more properties of a molecular metal ligand in a sample, comprising the steps of: providing a quantum sensor; exposing the quantum sensor to the sample; applying an illumination signal to the quantum sensor for a first predetermined duration; and detecting a photoluminescence intensity emitted from the quantum sensor; wherein a characteristic of the detected photoluminescence intensity is indicative of one of the properties of the molecular metal ligand in the sample.

In some embodiments, the applied illumination signal comprises optical illumination having a wavelength in a range of about 415 nm to about 630 nm, preferably about 480 nm to about 560 nm and more preferably about 532 nm.

In some embodiments, the applied illumination signal may comprise a pulsed signal having an excitation phase of about 200 ns to about 100 μs, preferably about 2 μs to about 20 μs and more preferably about 5 μs. Typically, this is followed by an interaction time during which excited electron spins have time to recover before the next illumination pulse is applied. The interaction time between excitation phases may range from about 12 ns to about 10 ms.

In some embodiments, the photoluminescence intensity is detected at one or more wavelengths in a range of about 620 nm to about 850 nm, preferably between about 637 nm to about 800 nm. The detected photoluminescence intensity is ideally measured during an excitation phase of the illumination signal applied to the quantum sensor.

In some embodiments the method comprises the step of measuring rate of decay of the detected photoluminescence intensity, wherein the rate of decay (1/T₁) indicates a property of the sample corresponding to loading factor of the molecular metal ligand.

In some embodiments the method comprises the step of comparing the measured rate of decay of the detected photoluminescence intensity with a background rate of decay measured from the quantum sensor when the illumination signal is applied in the absence of the sample.

The method may be deployed using a broad range of molecular metal-ligands or metal binding molecules such as but not limited to metalloproteins, metal-chelating agents and other metal-binding agents. It may be preferred that these molecules have a superparamagnetic core. In one embodiment, the metalloprotein is ferritin and the determined property may be loading factor which is indicative of iron bound to ferritin in the sample.

In some embodiments, the property is loading factor, which is indicative of an extent of loading of a metal or metalloenzyme within the molecular metal ligand or metal binding molecule. The metals may include but are not limited to e.g. vanadium, manganese, iron, cobalt, nickel, copper, gadolinium, and cadmium.

The quantum sensor may comprise an addressable spin defect in a semiconductor material. In some embodiments, the spin defect is a nitrogen-vacancy (NV) defect that has been engineered in the semiconductor material. The semiconductor material may be provided in any suitable form such as, but not limited to: a single bulk semiconductor chip, a two-dimensional semiconductor layer, and low dimensional semiconductor nano particles, to name a few. In some embodiments, the semiconductor comprises an engineered diamond and may be produced via a range of processes such as e.g. chemical vapour deposition (CVD) or high-pressure-high-temperature (HPHT) processes although other semiconductor materials such as silicon carbide are also contemplated.

In some embodiments in which the quantum sensor comprises a semiconductor material, the quantum sensor may comprise one or both of:
(a) NV defects at a density of from about 0.001 to about 500 parts-per-million relative to the semiconductor site density, preferably about 1 part-per-million; and
(b) NV defects located less than about 100 nm, preferably less than about 20 nm, more preferably less than about 10 nm from the semiconductor material surface.

In some embodiments, the quantum sensor may comprise a plurality of quantum sensor elements, wherein exposing the quantum sensor elements to the sample comprises providing the quantum sensor elements in suspension within a fluid comprising the sample. The plurality of quantum sensor elements may have a range of different geometries and may be homogenous or heterogeneous in the geometry types across the plurality of quantum sensor elements. Some geometries comprise e.g. randomly shaped chunks, and non-random geometrical shapes such as cubes and other polyhedrons, spheres, discs and single crystal elements. In some embodiments, spheres and discs may be preferred for sensitivity and consistency of measurements. In some embodiments, the plurality of quantum sensor elements may each have a diameter of between about 20 nm and about 500 nm, preferably between about 50 nm and about 200 nm.

The sample may be any sample of interest such as an industrial sample or a biological sample. Preferably, the sample is a fluid sample such as a biological fluid sample. Examples of biological fluid samples may include but are not limited to: blood, blood serum, blood plasma, cerebrospinal fluid, urine, saliva, pericardial fluid, pleural fluid, synovial fluid, amniotic fluid, seminal fluid, sweat and tears.

In some embodiments, the method comprises one or more steps for preparing a fluid sample, such as the biological fluid sample, by performing one or more of centrifuging the fluid, heating the fluid, passing the fluid through a liquid chromatograph or selective membrane, modifying pH and performing immuno or affinity capture to simplify the fluid for determining properties of one or more target species within the biological fluid sample.

In some embodiments, the photoluminescence intensity may be detected using a CCD (charge-coupled device) or a complementary metal oxide semiconductor (sCMOS). In some embodiments, the detected photoluminescence intensity may convey information about uniformity of the sample with respect to the quantum sensor, particularly when the quantum sensor includes a single bulk semiconductor chip. Alternatively/additionally, the photoluminescence intensity may detected using a photodiode such as e.g. a silicon photodiode or an avalanche photodiode.

Viewed from another aspect, the present disclosure provides for use of the method according to the above aspect in an apparatus, system or protocol for diagnosing one or more of iron deficiency, iron deficiency anaemia, iron overload (including but not limited to hereditary haemochromatosis and transfusional iron overload) and inflammation. Such use of the system may comprise determining iron load in ferritin.

Viewed from another aspect, the present disclosure provides for use of the above method in a system or apparatus for guiding management and/or treatment of one or more of iron deficiency, iron deficiency anaemia, iron overload (including but not limited to hereditary haemochromatosis and transfusional iron overload) and clinically diagnosed inflammation.

Viewed from another aspect, the present disclosure provides a system for detecting one or more properties of a molecular metal ligand in a sample, the system comprising:
(a) a quantum sensor configured to be exposed to the sample;
(b) an illumination source configured to apply an illumination signal to the quantum sensor;
(c) a detector configured to detect a photoluminescence intensity emitted from the quantum sensor; and
(d) a controller configured to control operation of the illumination source to deliver pulsed illumination;
wherein a characteristic of the detected photoluminescence is indicative of one of the properties of the molecular metal ligand in the sample.

In some embodiments, the controller controls operation of the illumination source to deliver pulses of illumination having a duration of about 200 ns to about 100 μs, preferably about 2 μs to about 20 μs and more preferably about 5 μs.

In some embodiments, the controller controls operation of the illumination source to space pulses of illumination by a time duration from about 12 ns to about 10 ms. The illumination signal may comprise a sequence of pulses of illumination of equal duration, or the signal may provide a first sequence of pulses of equal duration followed by a second sequence of pulses of different equal duration.

In some embodiments, the controller may control operation of the illumination source to divert a beam path away from the quantum sensor between pulses of illumination. Alternatively/additionally the controller may control operation of the illumination source to extinguish illumination between pulses of illumination.

In some embodiments, the controller controls operation of the detector to detect photoluminescence intensity during an excitation phase of the pulsed illumination.

In some embodiments, the controller includes or is in operable communication with a processor configured to determine rate of decay (1/T₁) of the detected photoluminescence intensity, wherein the determined rate of decay indicates a property of the sample corresponding to loading factor of the molecular metal ligand. In some embodiments, the processor may be configured to compare the measured rate of decay with a background rate of decay measured from the quantum sensor when the illumination signal is applied in the absence of the sample.

In some embodiments, the processor may be configured to determine automatically, from a mathematical model, calibration curve or lookup table stored in memory associated with the processor, loading factor for the molecular metal ligand, wherein the calibration curve or lookup table associates values of rate of decay with values for loading factor. In some embodiments the mathematical model may represent a plurality of physical properties of the molecular metal ligand. In other embodiments, the mathematical model may approximate a relationship between values of rate of decay and loading factor in experimental data obtained from one or more samples of the molecular metal ligand.

In some embodiments, the processor may be configured to: receive or determine a value representing concentration of the molecular metal ligand in the sample; and determine automatically a concentration of a target species within the sample by multiplying a value representing the loading factor with the value representing the concentration of the molecular metal ligand.

In some embodiments, the illumination source is configured to emit illumination at one or more wavelengths in a range of about 415 nm to about 630 nm, preferably in a range of about 480 nm to about 560 nm, and more preferably about 532 nm.

In some embodiments, the quantum sensor comprises an addressable spin defect in a semiconductor material. In some embodiments, the semiconductor material may comprise a diamond which may be produced via any suitable method such as chemical vapour deposition (CVD) or high-pressure-high-temperature (HPHT) processes although other semiconductor materials such as silicon carbide are also contemplated.

In some embodiments, the spin defect is a nitrogen-vacancy (NV) defect that has been engineered in the semiconductor material.

In some embodiments, the quantum sensor may comprise a plurality of quantum sensor elements, and the system may include a vessel for providing the quantum sensor elements in suspension within a fluid comprising the sample.

The plurality of quantum sensor elements may have a range of different geometries and may be homogenous or heterogeneous in the geometry types across the plurality of quantum sensor elements. Some geometries comprise e.g. randomly shaped chunks, and non-random geometrical shapes such as cubes and other polyhedrons, spheres, discs and single crystal elements. In some embodiments, spheres and discs may be preferred for sensitivity and consistency of measurements.

In some embodiments, the plurality of quantum sensor elements may each have a diameter of between about 20 nm and about 500 nm, preferably between about 50nm and about 200 nm.

The sample may be any sample of interest such as an industrial sample or a biological sample. Preferably, the sample is a fluid sample such as a biological fluid sample. Examples of biological fluid samples may include but are not limited to: blood, blood serum, blood plasma, cerebrospinal fluid, urine, saliva, pericardial fluid, pleural fluid, synovial fluid, amniotic fluid, seminal fluid, sweat and tears.

In some embodiments, the detector comprises one or more of a charge-coupled device (CCD), a complementary metal-oxide-semiconductor (CMOS) sensor, and a photodiode such as a silicon photodiode or an avalanche photodiode.

In some embodiments, the quantum sensor, the illumination source and the detector are contained in an optically sealed housing that prevents incursion of light from outside the housing while in use.

### BRIEF DESCRIPTION OF DRAWINGS

The present invention will now be described in greater detail with reference to the accompanying drawings. It is to be understood that the embodiments shown are examples only and are not to be taken as limiting the scope of the invention as defined in the claims appended hereto.

FIG. 1 is a flow chart illustrating steps in a method of determining one or more properties of a molecular metal ligand or metal binding agent according to an embodiment of the disclosure

FIG. 2 is a schematic illustration representing a pulse sequence of the illumination signal according to an embodiment of the disclosure.

FIG. 3 represents a typical spin lattice relaxation (T1) measurement curve taken from an ensemble of nitrogen vacancy (NV) centres in diamond.

FIG. 4A is schematic illustration of a system for detecting one or more properties of a molecular metal ligand using a single crystal diamond quantum sensor according to an embodiment of the disclosure.

FIG. 4B is a schematic illustration of a system for detecting one or more properties of a molecular metal ligand using a quantum sensor according to another embodiment of the disclosure utilizing an ensemble of quantum sensor elements.

FIG. 5 is a schematic illustration showing the NV defect in diamond.

FIG. 6 represents the energy level scheme of the C₃ᵥ-symmetric NV defect in diamond.

FIG. 7 is a plot depicting spectroscopic aspects of the quantum sensing technique according to embodiments of the disclosure.

FIG. 8 is a dynamic light scattering measurement of 100 nm nanodiamond suspension.

FIG. 9 shows schematically the rapid magnetisation reversal of the super-paramagnetic ferritin core in a liquid sample of serum ferritin.

FIG. 10 shows simulations of the spatial distributions of ferritins on the diamond surface and NV centres throughout the nanodiamond. The shading represents the resulting relaxation times (T1) of the NV spins for 50 nm, 100 nm and 200 nm spherical nano sensing elements (nanodiamonds).

FIG. 11 shows schematically, interaction between ferritin and a quantum sensor according to an embodiment of the disclosure.

FIG. 12 shows the change in relaxation rate T1 for samples containing ferritin with variable iron load after 4 hours of interaction, where solid line A represents data fitted using a composite model, tight dash line B represents contributions from ferritin containing multiple subcores and wide dash line C represents ferritin containing single cores only.

FIG. 13 is a probability distribution of ferritin containing single cores(S) and multiple subcores (M) as a function of iron load.

FIG. 14 represents various values for T1 and iron load for a number of nucleation sites Nc within each ferritin.

FIG. 15 shows a curve D that has been fit using a 5th order polynomial to the data in FIG. 12 for a region of interest.

### DETAILED DESCRIPTION

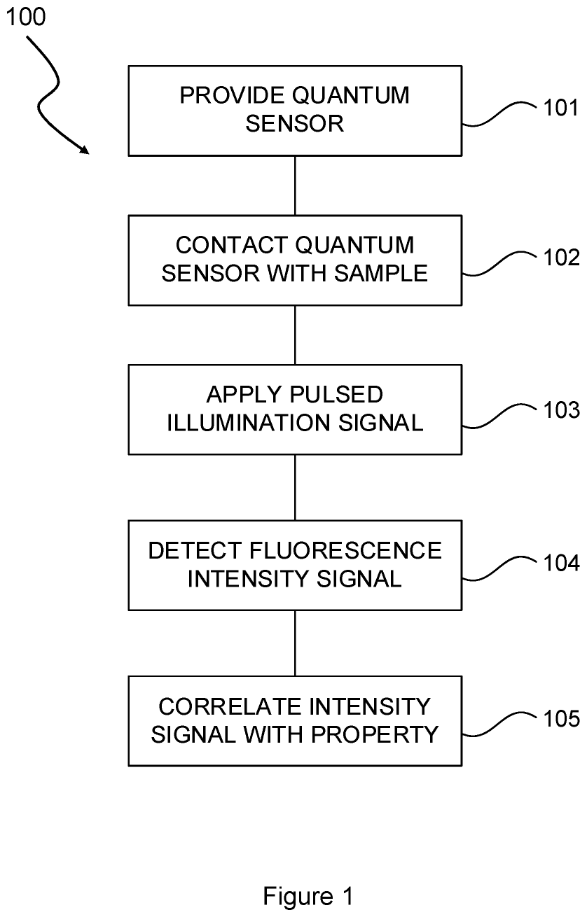

Referring firstly to FIG. 1, a flow chart shows steps in a method 100 of determining one or more properties of a molecular metal ligand or metal binding agent according to an embodiment of the disclosure. In a step 101 a quantum sensor is provided and in a step 102, the quantum sensor is exposed to the sample. Exposing the quantum sensor to the sample may involve bringing the sensor into sufficiently close proximity to or making contact with the sample (or vice versa) to permit interaction between the molecule and the quantum sensor which is measurable using the techniques disclosed herein. In a step 103, an illumination signal is applied to the quantum sensor for a predetermined duration. Typically, the illumination signal is a pulsed signal. In a step 104 a photoluminescence intensity emitted from the quantum sensor is detected which is indicative of one of the properties of the molecular metal ligand in the sample.

In a preferred embodiment disclosed herein, the molecular metal ligand is a metalloprotein such as ferritin which the body's main iron store. Although concentration of ferritin in the blood may provide an indirect correlation of iron stores in healthy individuals, chronic diseases and acute inflammation are known to increase ferritin concentration in the blood but not necessarily the quantity of ferritin-bound iron. Therefore, in preferred embodiments, the property of interest is the loading factor of iron atoms present in ferritin since it is the iron load of ferritin in the body that is determinative of iron stores.

Loading factor may be expressed in numerous terms which provide a representation of the amount of iron bound to ferritin within the sample. For example, loading factor may be expressed as an absolute number of bound iron atoms, or a percentage or other qualitative expression corresponding to the binding capacity occupied by iron e.g. where 0% loading factor corresponds to zero bound iron and 100% loading factor corresponds to a maximum load of ˜4500 iron atoms per ferritin. In some embodiments, the loading factor is a qualitative term used to convey the extent of saturation of the ferritin with iron, for instance the loading factor may be indicated as “low” and at risk for iron deficiency, “normal” or “high” and at risk for iron overload, and these terms may be indicated when the loading factor determined for a sample falls within certain predetermined ranges. The ranges for qualitative expression of loading factor may be determined based on a population of individuals having a broad range of traits (such as e.g. known medical or hereditary conditions, lifestyle indicators, age or the like), or based on a subset of individuals having particular traits and/or diagnosed conditions known to affect iron loading. In some embodiments, the ranges may be personalised, recognising that a loading factor which may be too high or low to fall within the “normal” range for some populations may be “normal” for some individuals or populations of individuals.

It is to be understood, however, that the present disclosure is not to be limited to the determination of iron load in ferritin, and that the method may be used to determine loading factor of other metals (or metalloenzymes) that may be bound to the molecular metal ligand or other metal binding agent contained within the sample.

As will become apparent in the parts that follow, the quantum sensor may comprise a single or ensemble of engineered defects in a semiconductor material. In a preferred embodiment the engineered defects include NV centres in diamond. The quantum sensor may be comprised of engineered defects in a single bulk semiconductor chip or engineered defects in small sensor elements each having one or multiple defects. Owing to the relatively high price of NV diamond sensing chips, and the undesirability of re-using chips particularly to test biological fluids, a disposable quantum sensor may be preferred. To address these issues, preferred embodiments of the present disclosure may utilise an ensemble of small NV diamond quantum sensor elements (hereinafter nanodiamonds) that can be put into suspension within a sample fluid prior to illumination. Use of nanodiamonds provides a larger surface area for attachment of ferritin (and/or metal ligand and/or metal binding molecule) improving measurement sensitivity in some embodiments by up to 8 or 9 orders of magnitude compared to other electron spin based approaches such as MRI.

The illumination signal is pulsed to control the initialisation and readout of NV centres. The duration of pulses may be from 200 ns to about 100 μs, preferably about 2 to 20 μs and more preferably about 5 μs to balance readout noise and efficient initialisation of NV centres.

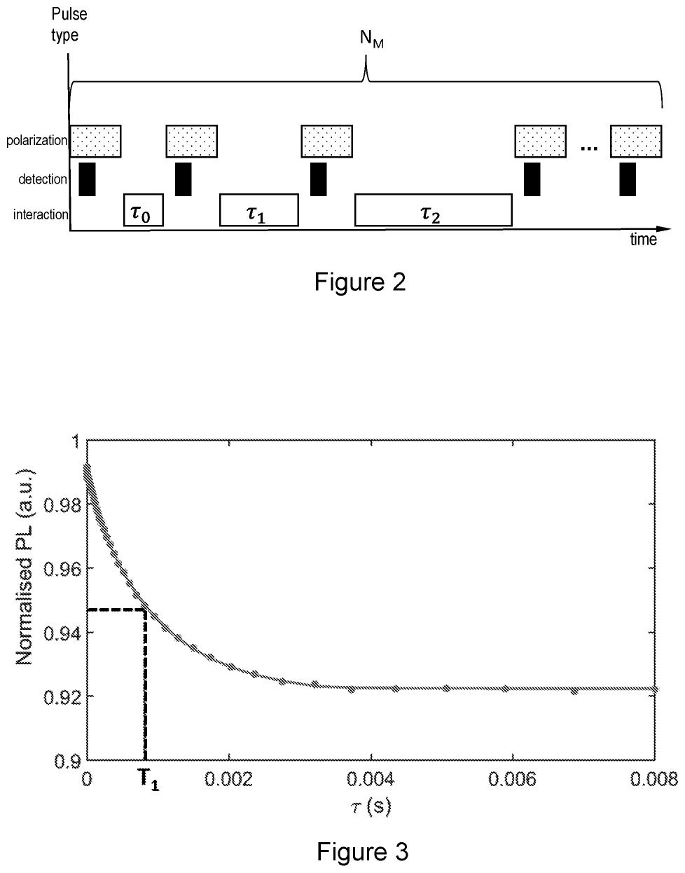

As detailed in FIG. 2, the measurement involves optical polarization into the |0 [symbol] state (top row of pulses, labelled “polarization”), followed by an interaction period of the NV with the target species (which, in the case of ferritin, arises from the presence of iron in the ferritin core) for a range of interaction times, τ. The middle row of pulses (labelled “detection”) represent the time intervals over which the photoluminescence intensity signal is measured. The sequence is completed by reapplication of the polarizing illumination (second pulse in the polarization/illumination signal) which acts as both a readout method (causing photoluminescence which is indicative of the NV spin state) and repolarizing the system for the next measurement. This process is repeated some N_{M} times (with a practical range of 1≤N_{M}≤10⁷, but typically N_{M}˜105) in order to increase the signal to noise ratio of the measurement.

The effect of the target species is inferred by measurement of the deviation of the NV quantum state from its initial state during their interaction. Possible approaches to quantifying this relaxation time include measuring the free-induction decay time (typically denoted T₂^{*}); the relaxation time in a Rabi experiment (typically denoted T₂^{R}); the relaxation time under pulsed microwave dynamical decoupling protocols (including but not limited to spin-echo and Carr-Purcell-Meiboom-Gill (CPMG) multiple microwave pulse sequence), or X-Y spin relaxation time (typically denoted T2 or T₂); or the longitudinal spin relaxation time (typically denoted T1 or T₁). Longitudinal relaxation times are typically employed as these offer the longest baseline signal, and avoid the need to apply microwave control of the NV. Under this approach, the characteristic time it takes for the NV |0 [symbol] state population to relax to 1/e of its initial value is referred to as the T₁ time.

FIG. 3 shows a typical T1 measurement curve, taken from an ensemble of NV centres. It is characterised by an exponential decay in photoluminescence (normalised) due to the loss of polarisation brought about by environmental spin noise. T1 curves may be obtained using the pulse sequence of FIG. 2. During free evolution time (also referred to as the interaction time)τ, the NV centre experiences longitudinal relaxation.

As noted above, with the exception of the initial polarisation phase, subsequent polarisation phases of the illumination signal simultaneously facilitate optical determination of the spin state of the NV centres and re-polarisation of the NV ensemble. The optimal readout pulse duration is dictated by the laser power intensity and the sensing volume. In preferred embodiments, illumination pulses of a duration of between 200 ns and 100 μs are sufficient to re-polarise NV ensembles within a sensing volume of 2.5×10⁻³ mm² and incident optical power intensity at 532 nm of 240 W/mm².

In some embodiments, it may be preferable to obtain a background signal which can be used to remove background noise from the measurement. This may be achieved by applying the measurement sequence to the quantum sensor in the absence of the sample and determining the corresponding background rate of decay. The measured rate of decay of the photoluminescence signal obtained from the quantum sensor in the presence of the sample can then be compared with the background rate of decay to determine the effects that are attributable to the sample. In some embodiments, the background rate of decay is subtracted from the rate of decay of the photoluminescence obtained in the presence of the sample to remove background contribution.

In order to detect properties of the molecular metal ligand at low levels (e.g. low concentrations), it may be desirable to manipulate the sample. Various chemical or biochemical strategies may be utilized to prepare the sample for analysis, including according to techniques disclosed herein. In the case of determining iron load, it is possible to exploit ferritin's inherent biophysical properties to achieve simple, cost effective sample enrichment. Ferritin is a very stable protein that has been found to be unaffected by heat treatments that denature the majority of serum proteins. The iron content also remains undisturbed. Therefore heating the sample to between about 65° C. and about 85° C., preferably to about 70° C., may assist with assessment of the sample. A subsequent centrifugation step could be utilised to pellet the insoluble protein aggregates leaving the soluble ferritin in solution, which can then proceed to detection and analysis utilizing the quantum sensor. Other options for sample preparation include passing a liquid sample through a liquid chromatograph or a selective membrane, modifying pH and performing immuno or affinity capture to simplify the fluid.

In preferred embodiments, the applied illumination signal has a wavelength in a range of about 415 nm to about 630 nm, preferably 480 nm to about 560 nm and usually about 532 nm which conveniently corresponds to commercial green laser and LED light sources. For detection of the photoluminescence intensity, it is preferable to detect at one or more wavelengths in a range of about 620 nm to about 850 nm, such as about 637 nm to about 800 nm, to cover the zero phonon line (ZPL) of the NV centres and the stokes shifted phonon sideband fluorescence which conveniently corresponds to red light in the visible-near-infrared spectrum. The photoluminescence intensity may be detected using any suitable means. In some embodiments, this may include use of a sensitive CCD camera preferably a scientific complementary metal oxide semiconductor (sCMOS). Use of a CMOS detector in the case of a quantum sensor comprising a single crystal quantum sensor (as opposed to a plurality of nanodiamond quantum sensor elements) may have additional utility in ascertaining uniformity of the biological sample with respect to the quantum sensor. In some embodiments, the photoluminescence intensity may be detected using one or more photodiodes such as a silicon or avalanche photodiode.

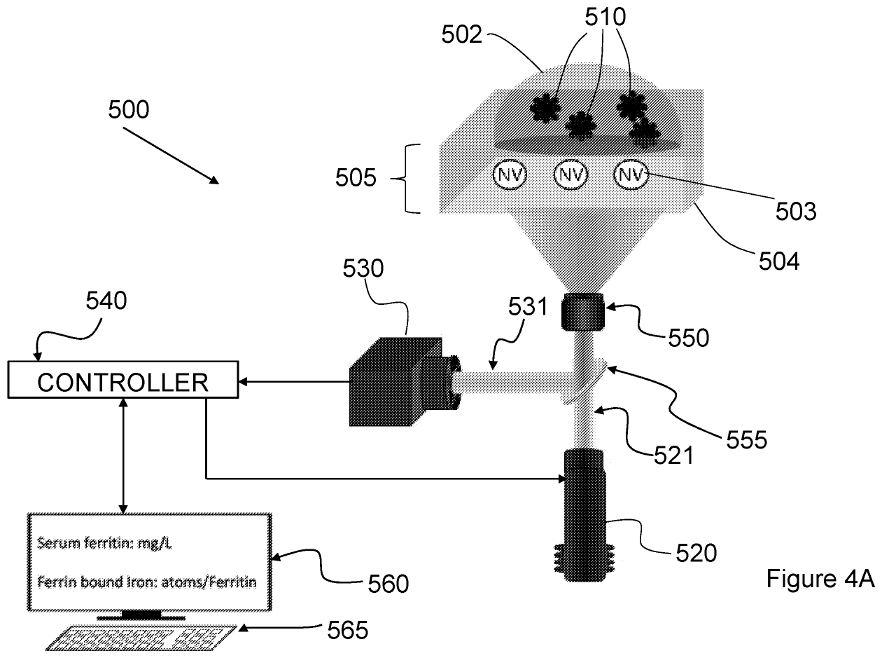

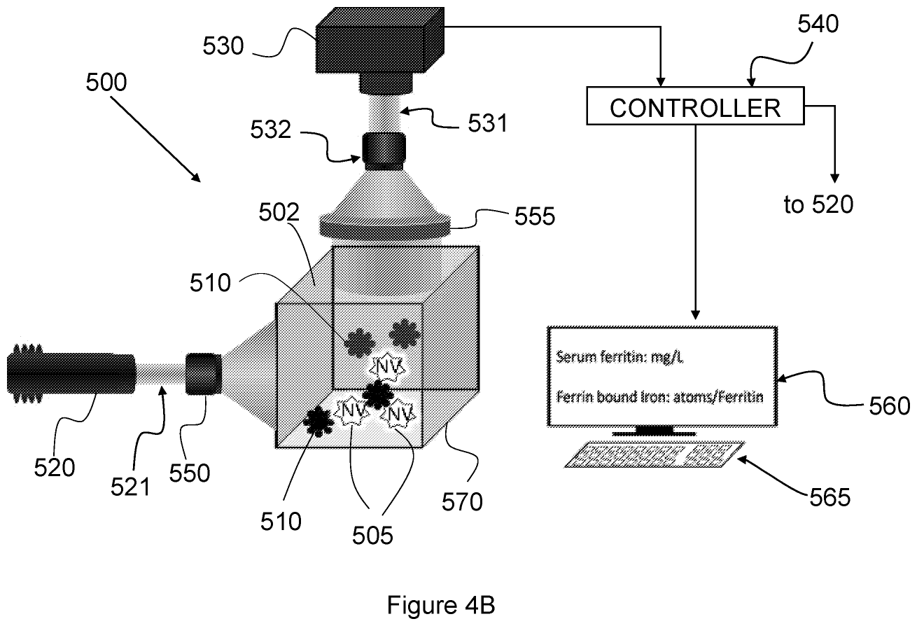

FIG. 4A is schematic illustration of a system 500 for detecting one or more properties of a molecular metal ligand 510 (e.g. ferritin) in a sample 502. The sample 502 is applied to or contacted with a quantum sensor 505 comprising a single diamond substrate 504 containing a plurality of NV defects 503. An illumination source 520 applies an illumination signal 521 (ideally green light in the visible spectrum e.g. at about 532 nm) to the quantum sensor 505 with the sample 502 and a detector 530 (e.g. a CMOS or photodiode) detects the photoluminescence intensity emitted from the quantum sensor. In the case of ferritin, the detector 530 detects red light in the visible spectrum, ideally between about 650 nm and about 800 nm. A lens 550 may focus the illumination signal 521 on the sample 502 and, a mirror or bandpass filter 555 may be used to direct the emitted photoluminescence 531 to detector 530. A controller 540 controls operation of the illumination source 520 to deliver pulsed illumination e.g. having the pulse sequence of FIG. 2. A characteristic such as rate of decay of the detected photoluminescence 531 is indicative of one of the properties of the molecular metal ligand 510 in the sample 502. The characteristic may be presented on a user interface which may comprise a visual display device or monitor 560 and optional keyboard 565 for a user to enter details about the sample such as concentration of the target molecule.

In preferred embodiments, controller 540 controls operation of the illumination source 520 to deliver pulses of illumination having an excitation phase of about 200 ns to about 100 μs, preferably about 2 μs to about 20 μs and more and preferably about 5 μs, e.g. for a sensing volume of 2.5×10⁻³ mm² and incident optical power intensity at 532 nm at 240 W/mm². Ideally, controller 540 controls the illumination source 520 so that pulses are separated by variable time durations from about 12 ns to about 10 ms. This may be achieved e.g. by the controller 540 causing the beam path of the illumination signal 521 to be diverted away (or obscured) from the quantum sensor 505. This may be achieved by modulation of the illumination source or by using e.g. an acousto-optic modulator (AOM, also referred to as a Bragg cell or acousto-optic deflector, AOD) or a mechanical chopper, pocket cell or other suitable means. Alternatively, controller 540 may cause emissions from the illumination source 520 to be extinguished between excitation pulses.

As disclosed herein, controller 540 controls operation of the detector 530 to detect a photoluminescence intensity during an excitation phase of the pulsed illumination signal 521. Thus, the controller may receive a photoluminescence intensity signal detected only during excitation phases, or the controller may receive a continuous photoluminescence intensity signal and utilize the control signal used to control the illumination source 520 to select only the detected photoluminescence intensity signal segments that corresponds to excitation phases of the pulsed illumination signal.

In preferred embodiments, controller 540 includes a processor configured to determine a rate of decay of the detected photoluminescence intensity, wherein the determined rate of decay indicates a property of the sample 502 corresponding to loading factor of the molecular metal ligand 510 (e.g. ferritin). In some embodiments, the processor is configured to compare the measured rate of decay with a background rate of decay measured from the quantum sensor 505 when the illumination signal is applied in the absence of the sample to eliminate background signal not attributable to interaction between the molecular metal ligand and the quantum sensor. Ideally, the processor is configured to determine automatically, from a function or lookup table stored in memory associated with the processor of the controller 540, loading factor for the molecular metal ligand, wherein the function or lookup table associates values of rate of decay with values for loading factor. The function (such as a calibration curve) or lookup table may be based on defined standards or data obtained from previously measured samples. Suitable approaches for defining functions used to determine loading factor from T1 values are described herein and include i) using a physical model that describes how the magnetic behaviour evolves as a function of loading factor, and ii) using a non-physical model such as a mathematical model which may be determined by fitting a curve to experimental data. Curve fitting may be achieved by manual approximation or by use of curve-fitting software or algorithms as are known by those skilled in the art. The values of the loading factor can then be presented to the user e.g. on monitor 560 or in a report generated by the controller 540 and transmitted by conventional means (including e.g. computer networks) to other devices.

FIG. 4B a schematic illustration of a system 500 for detecting one or more properties of a molecular metal ligand 510 (e.g. ferritin) in a sample 502. Corresponding features of the system of FIG. 4A are identified with like numerals. A second lens 532 may be provided to focus the emitted photoluminescence on the detector 530. The system may include other optical elements such as filters, lenses, polarisers and reflectors to enhance operation of the system as would be understood by one of skill in the art. It will be noted that in FIG. 4B, the quantum sensor is provided in the form of a plurality of quantum sensor elements 505 in a suspension with the liquid sample 502 contained within a vessel 570. Use of a bulk ensemble of quantum sensor elements 505 increases the available surface area for ferritin attachment and hence measurement sensitivity.

Embodiments of present disclosure provide techniques, embodied in the form of a method and system, for clinical quantification of loading factor within a molecular metal ligand or other metal binding agent using a new assaying technique that uses optical excitation and measurement to determine a change in the behaviour of the quantum sensor due to the properties of the molecular metal ligand in the sample. Specifically the optical technique exploits the different photoluminescence intensities emitted by atomic defects in the quantum sensors when illuminated, to determine the spin state. The rate of decay of the photoluminescence signal can then be used to determine the change in relaxation time induced by e.g. the presence of iron in the ferritin core, and the magnitude of the change is indicative of the quantity of iron bound within the molecule. Thus, the rate of decay may be used to provide a measure of the change in relaxation time of electron spins (T₁) in the quantum sensor in the presence of a metal, metalloprotein, metal-molecular complex or metal ligand.

While the invention utilises principals of physics also used in magnetic resonance imaging (MRI), the sensitivity of traditional MRI methods such as FerriScan is limited by the large standoff distance of the magnetic pick up coils. The present disclosure provides an approach which can achieve considerably greater sensitivity by utilising quantum sensors/quantum sensor elements having NV defects manufactured within the diamond at a location that is much closer to the sample being analysed. In some embodiments, the sensing defects can be engineered less than 10nm from the surface of the diamond, making the quantum sensor up to nine orders of magnitude more sensitive than traditional MRI. This enables testing on small, low concentration samples such as blood serum. Embodiments of the present disclosure may be capable of measuring ferritin bound iron at physiologically relevant concentrations as low as or less than 30 ng/ml, thus enabling a clinical assay for direct detection of iron load in serum ferritin and other body fluids. It is to be understood that in addition to detecting properties in liquid samples, other samples including tissue homogenates and/or extracts could also be used particularly if capable of being prepared into a liquid form for testing.

For more complete understanding of the sensing techniques disclosed herein, an overview of the analytical framework follows.

### Quantum Relaxometry with NV Centres in Diamond

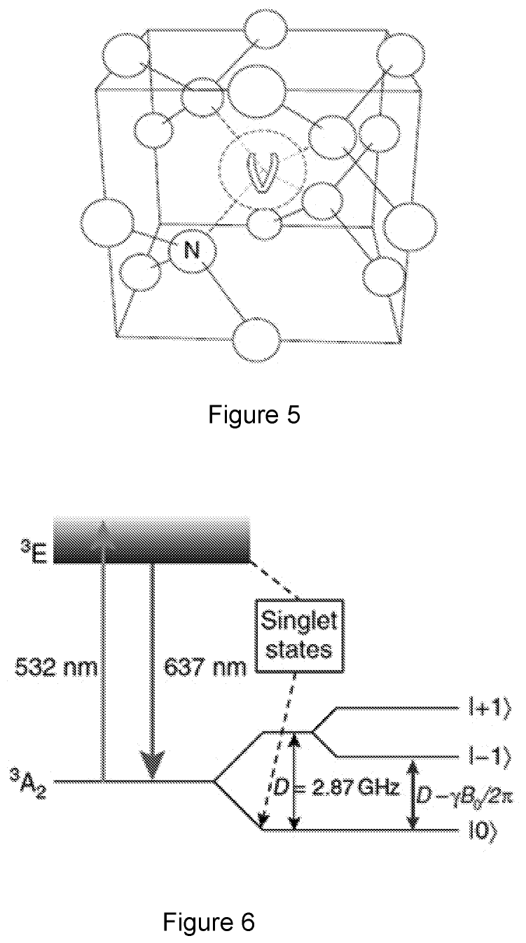

FIG. 5 is a schematic illustration showing the nitrogen-vacancy (NV) centre point defect in diamond, comprised of a substitutional nitrogen atom (N) and an adjacent crystallographic vacancy (V). Blank spheres represent carbon atoms. This structure forms the basis of the material platform utilised for the quantum sensor of this disclosure. Advantageously, the NV diamond quantum sensor exhibits a beneficial combination of spin-state controllability, quantum state robustness, and room temperature operation.

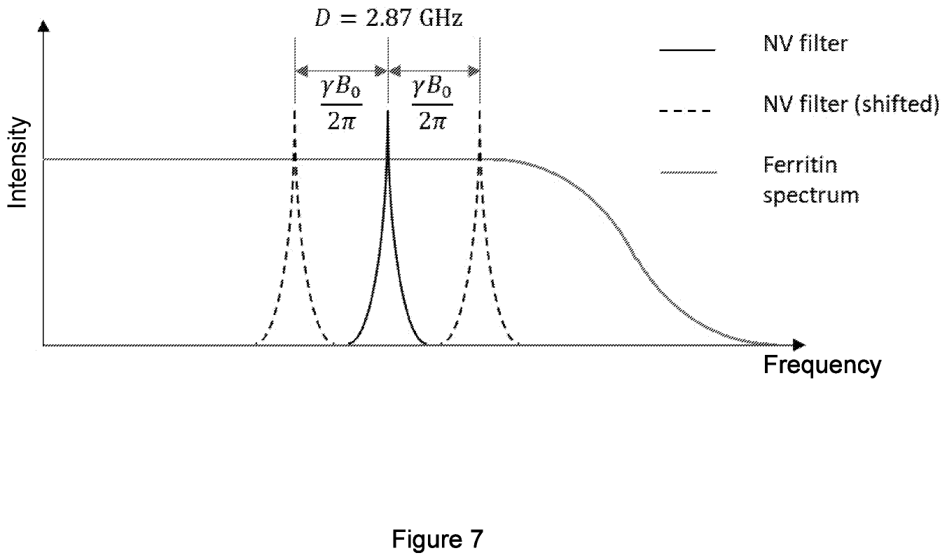

The energy level scheme of the C₃ᵥ-symmetric NV system is represented in FIG. 6. It consists of ground (³A₂), excited (³E) and singlet electronic states. The ground-state spin-1 manifold has three spin sublevels (|0 [symbol] , |±1)), which at zero-field are split by D=2.87 GHz. Since these sublevels have different photoluminescence intensities upon illumination, it is possible to determine spin-state by optical measurement. For example, upon optical excitation at 532 nm, the population of the |0 [symbol] state may be read out by monitoring the intensity of the emitted red light. FIG. 7 is a plot depicting the spectroscopic aspects of this technique. The NV filter function (solid black line) isolated the regions of the ferritin spectrum (grey) that are close to the NV spin transition frequency. The NV filter can be shifted (broken line) by tuning the strength of an applied external magnetic field, which generally allows for species exhibiting narrow spectral features to be resonantly targeted. It is to be noted however, that such an approach is not needed for the case of ferritin, owing to the ˜10 GHz width spectrum, which is at least four orders of magnitude greater than that of the filter function.

The degeneracy between the |+1 [symbol] and |−1 [symbol] , states may be lifted with the application of an external magnetic field, B₀, with a corresponding separation of 2γB₀, (where γ=17.6×10⁶ rad s⁻¹G⁻¹ is the NV gyromagnetic ratio), permitting all three states to be accessible via microwave control. By isolating either the |0 [symbol] =↔|+1 [symbol] or |0 [symbol] ↔|−1) transitions, the NV spin system constitutes a controllable, addressable spin qubit. Similar to above, it is to be noted that this is not necessary for the ferritin case due to its broad spectrum.

Much attention has been focused on using measurements of the NV quantum phase interference between its spin sublevels or dephasing rates to characterize dynamic processes occurring in external magnetic environments. These protocols have been shown to have remarkable sensitivity to frequencies in the kHz-MHz range, and are thus well suited to characterizing nuclear spin environments. However, to achieve the desired sensitivity to the more rapidly fluctuating (GHz) fields associated with electron spin environments, and more importantly, the ability to be frequency-selective, complex and technologically challenging pulse sequences would be necessary.

Alternatively, if a transition frequency of an environmental spin approaches that of the NV spin, they will exchange magnetization and their longitudinal spin relaxation rates will increase. This enables the detection of environmental spins by monitoring changes in NV T1 times, where the T1 is defined as the evolution time at which the population of the ground state |0 [symbol] is 1/e times its initial value. This is inferred by the difference in photoluminescence intensity between the |0 [symbol] and |±1 [symbol] states. This approach can be more sensitive because NV T1 times can be up to three orders of magnitude longer than spin-echo-based T2 times. Advantageously, owing to the wide NV quantum sensor zero-field splitting, transitions in the ground-state spin triplet manifold are far off-resonance from the MHz transition frequencies of the chief magnetic defects in diamond (nitrogen electron-donor defects, referred to hereafter as P1 centres, and ¹³C nuclei), leaving NV transitions unable to be excited unless brought into resonance using an axial magnetic field (B₀˜512 G for an electron spin).

The weak spin-orbit coupling to crystal phonons (and low phonon occupancy, owing to the large Debye temperature of diamond) leads to longitudinal relaxation of the spin state on timescales of roughly T1˜1-10 ms at room temperature, and hundreds of seconds when limited by NV-NV cross-relaxation at temperatures below ˜20 K. On the other hand, the transverse relaxation time (T2) of the NV spin is the result of dipole-dipole coupling to other spin impurities in the diamond crystal, which may be utilised for this method. Hence, the relatively long relaxation time of the NV ground state and inherent sensitivity to GHz frequencies, together with room temperature operation and optical readout, make it an ideal system for detection of the GHz magnetisation reversal rates associated with the iron load within ferritin.

Diamonds with a long intrinsic T1 time are preferred since changes to this value (e.g. due to the presence of ferritin) will be easier to measure. Advantageously, an easily detectable signal can be achieved by the techniques disclosed herein utilizing a quantum sensor comprising a type 1b, IIa or CVD diamond in some embodiments, particularly those utilising a single bulk diamond semiconductor chip. Type Ib diamonds are considerably cheaper than electronic grade type IIa diamonds which are commonly preferred for analytical applications. Viability of type 1b diamond for sensing purposes according to embodiments of the present disclosure represents a significant cost advantage that may support widespread uptake e.g. in routine laboratory testing.

In some embodiments, a controlled external magnetic field, B₀, would provide means to tune the transition energies of the NV quantum sensor, ω_{NV}=2πD+γB₀, into resonance with any desired target species for which the property (e.g. loading factor) is sought to be determined. In present case of ferritin however, the associated fluctuations are so rapid that the applied illumination signal is able to excite the NV ground state spin transitions even at zero magnetic field (as detailed below). This allows the quantum sensor to maintain a definitive insensitivity to other paramagnetic defects that may reside near or on the diamond, thus ensuring that the detected signal originates from ferritin exclusively.

In the following overview of the theoretical framework associated with techniques disclosed herein, a general and robust method to determine the average iron load from a plurality of ferritin is provided.

### Measurement Protocol and Performance Metrics

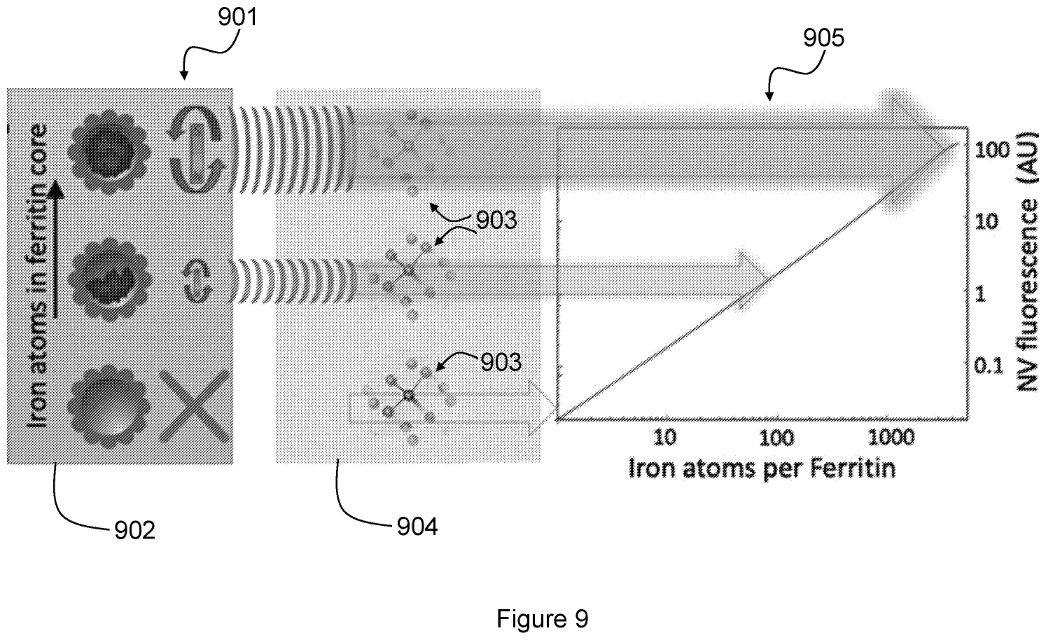

At zero-magnetic field, both NV transitions, |0 [symbol] =↔|+1 [symbol] and |0 [symbol] ↔|−1 [symbol] ,occur at ˜2.87 GHz. These transitions may be split symmetrically by the presence of an axial magnetic field and any axial NV-environment couplings. However, owing to the reversal rate of the ferritin core's magnetisation compared to any other timescale within the quantum sensor-ferritin system, these effects can be ignored. The component of the effective magnetic field produced by the ferritin core that is on resonance with the NV spin transition rate behaves as a transverse RF field, and is thus able cause the NV spin to tunnel between its ground state spin levels. FIG. 9 shows schematically the rapid magnetisation reversal (at 901) of the super-paramagnetic ferritin core in a liquid sample of serum ferritin 902 producing a randomly fluctuating radio frequency (RF) signal whose amplitude is determined according to the iron atoms per ferritin. NV defects 903 in the diamond quantum sensor 904 provide transduction of the RF signal amplitude into photoluminescence intensity under optical excitation. This enables quantification of the number of iron atoms per ferritin as depicted schematically in the graphic element at 905.

Whilst other paramagnetic impurities in the system do not result in any additional tunnelling between the NV spin states, their presence can result in fluctuations of the corresponding transition energies. Thus, to analytically model the response of the NV spin to the ferritin signal, it is necessary to account not only for the coupling between the NV and the ferritin core, but also the destruction of the phase coherence between the NV sublevels (dephasing, occurring at a rate of Γ₂) due to other spins present. Depending on the relative strengths of these processes (that is, how many energy exchanges may occur before the NV spin is dephased), the NV spin can exhibit diverse behaviour ranging from coherent (i.e. Rabi) oscillations to overdamped exponential decay. However, as there are typically more sources of dephasing than on-resonance signals, Γ₂>>B for the cases considered. Under this regime, if the NV is initially polarized in its |0 [symbol] state both the |0 [symbol] ↔|+1 [symbol] and |0 [symbol] ↔|−1) transitions will be excited, and the subsequent population at time t when coupled to a spin system of fluctuation rate ω will be:

$$P ( t ) = \frac{1}{2} + \frac{1}{2} exp ( - \frac{{B}^{2} {Γ}_{2} t}{2 ( {Γ}_{2}^{2} + {δ}^{2} )} ) \qquad ( Eq . 1 )$$

where δ=2πD−ω is the difference in transition frequencies between the NV (2πD=2π×2.87 GHZ) and the frequency of the target signal, ω. As such, the above relaxation rate effectively acts as a spectral filter of the ferritin signal about ω=2πD, and of width Γ₂.

For all cases of practical interest, the system in use comprises many NVs and many ferritins. The response of j^{th} NV spin will thus be the result of the effective field due to all ferritin cores to which it is coupled, and the resulting signal will be comprised of a distribution of frequencies, referred to hereafter as its spectral distribution, S (ω), which is essentially just a probability distribution for ω. This implies that the average relaxation rate is therefore found by averaging the relaxation rate (exponent) in Eq. 1 over the environmental spectrum as shown below in Eq. 2.

$${Γ}_{T} = \frac{1}{{T}_{1}} = \frac{{B}^{2}}{2} {\int }_{- ∞}^{∞} \frac{{Γ}_{2}}{{Γ}_{2}^{2} + {δ}^{2}} S ( ω ) d ω \qquad ( Eq . 2 )$$

The analytic form of the ferritin spectral power distribution, as dependent on the physical properties of its iron core, is determined below.

Owing to variation in diamond geometries, and NV centre placement within those diamonds, the NV ensemble will exhibit a distribution of coupling strengths to their proximate ferritin cores. Moreover, the loading factor will also exhibit some variation over the sample. As such, the total measured signal from all NVs present will depend on these distributions.

The full photoluminescent (PL) response for many NVs is a summation over all j individual NV signals present in the ensemble, each having optical contrast cⱼ and relaxation rate Γ_{T}^{(j)}

$$PL ( t ) = {\sum }_{j} [ 1 - {c}_{j} + {c}_{j} exp ( - {Γ}_{T}^{( j )} t ) ] + η ( t ) , \qquad ( Eq . 3 )$$

where η(t) is a stochastic signal describing the shot noise statistics associated with the PL measurement. If the quantum sensor consists of multiple diamonds, as in the nanodiamond case considered in this disclosure, summation over all NVs (j) may be decomposed into a sum over the NVs in each nanodiamond, 1<j<N_{NV}^{(m)}, each having optical contrast cⱼ and polarisation efficiency pⱼ; followed by a sum over all nanodiamonds, 1<m<N_{ND}, in the ensemble:

$$PL ( t ) = {\sum }_{m}^{{N}_{ND}} {\sum }_{j}^{{N}_{NV}^{( m )}} [ 1 - {c}_{j} + {c}_{j} {p}_{j} exp ( - {Γ}_{1}^{( j )} t ) ] + η ( t ) \qquad ( Eq . 4 )$$

This allows for explicit consideration of the variation in nanodiamond size and shape in evaluating sensor metrics, such as the sensitivity and coefficients of variation, for commercially available sources of nanodiamond.

Due to large variations in separation distances between the NV centres and the ferritin cores, evaluation of the general form of the PL signal in Eq. 4 will lead to extremely complex lineshapes and decay profiles that are highly dependent on the sensor geometry, thereby making a general approach to accurate fitting prohibitive. In particular, given that a significant proportion of NVs will reside within a few nanometres of a ferritin core, the short-time behaviour of the PL signal will be extremely sharp and thus highly sensitive to electronic timing jitter. In order to accommodate this, the PL signals may be integrated over time, giving the measurement:

$$\begin{matrix}M = \int PL ( t ) dt \\ = {\sum }_{m}^{{N}_{ND}} {\sum }_{j}^{{N}_{NV}^{( m )}} [ 1 - {c}_{j} + {c}_{j} {p}_{j} exp ( - {Γ}_{1}^{( j )} t ) ] + η ( t ) \\ = {M}_{0} + ϵ + c {\sum }_{m}^{{N}_{ND}} {\sum }_{j}^{{N}_{NV}^{( m )}} \frac{{p}_{j}}{{Γ}_{1}^{( j )}}\end{matrix} \qquad ( Eq . 5 )$$

Despite the total PL signal (referred to in Eq. 4) resembling a stretched exponential for times t<<1/Γ₀, it is comprised of a finite number of pure exponentials. As such, whilst the signal itself is complex and difficult to analyse, its time integral (as shown in Eq. 5) is independent of the shape of the decay profile. Rather, it depends only on average properties of molecules of intermediate size, such as nanoparticle geometry and impurity densities, and thus gives a decomplexified window into the ensemble averaged ferritin loading factor.

In order to decouple the effect of ferritin on the quantum sensor from the background signal, measurements of the nanodiamond ensemble with and without ferritin applied may ideally be compared, as follows.

First, a PL measurement is performed on the nanodiamond ensemble prior to contacting the sample to obtain a background (BG) signal:

$${M}_{BG} = {M}_{0} + {ϵ}_{BG} + c {\sum }_{m}^{{N}_{ND}} {\sum }_{j}^{{N}_{NV}^{( m )}} \frac{1}{{Γ}_{BG}^{( j )}} \qquad ( Eq . 6 )$$

Second, the sample is applied to the nanodiamond ensemble and a PL measurement is performed to obtain a target (BG+T) signal:

$${M}_{BG + T} = {M}_{0} + {ϵ}_{BG + T} + c {\sum }_{m}^{{N}_{ND}} {\sum }_{j}^{{N}_{NV}^{( m )}} \frac{1}{{Γ}_{BG}^{( j )} + {Γ}_{T}^{( j )}} \qquad ( Eq . 7 )$$

Third, the integrated difference between signals is evaluated:

$$\begin{matrix}Δ M = {M}_{BG} - {M}_{BG + T} \\ = {ϵ}_{Δ M} + c {\sum }_{m}^{{N}_{ND}} {\sum }_{j}^{{N}_{NV}^{( m )}} ( \frac{1}{{Γ}_{BG}^{( j )}} - \frac{1}{{Γ}_{BG}^{( j )} + {Γ}_{T}^{( j )}} )\end{matrix} \qquad ( Eq . 8 )$$

The above expression for ΔM involves summation over a large number of nanodiamonds, N_{ND}. For example, using a 1 mL blood sample, between 10⁶ and 10¹⁰ nanodiamonds would be required in order to ensure sufficient available diamond surface to bind all ferritin expected from physiologically relevant concentrations. As such, the distribution for the dispersion of ΔM is well approximated by a normal distribution with mean μ_{M} and spread σ_{M} given by:

$${μ}_{M} = 〈 Δ M 〉 = {N}_{ND} {μ}_{ND} \qquad ( Eq . 9 )$$

$${σ}_{M} = \sqrt{{N}_{ND}} {σ}_{ND}$$

Where N_{ND} is the total number of nanodiamonds in the ensemble, and μ_{ND}and σ_{ND} are the mean and standard deviations associated with an individual nanodiamond, as obtained from their size, shape, and NV and ferritin location distributions,

$${μ}_{ND} = 〈 {\sum }_{j}^{{N}_{NV}^{( n )}} ( \frac{1}{{Γ}_{BG}^{( j )}} - \frac{1}{{Γ}_{BG}^{( j )} + {Γ}_{T}^{( j )}} ) 〉 \qquad ( Eq . 10 )$$

$${σ}_{ND}^{2} = 〈 {( {\sum }_{j}^{{N}_{NV}^{( n )}} ( \frac{1}{{Γ}_{BG}^{( j )}} - \frac{1}{{Γ}_{BG}^{( j )} + {Γ}_{T}^{( j )}} ) )}^{2} 〉 - {μ}_{ND}^{2} + {ϵ}_{ND}^{2}$$

The resulting coefficient of variation (CoV) for the nanodiamond ensemble quantum sensor under this protocol is can then be determined by:

$$CoV ( {N}_{ND} ) = \frac{{σ}_{M}}{{μ}_{M}} = \frac{1}{\sqrt{{N}_{ND}}} \frac{{σ}_{ND}}{{μ}_{ND}} \qquad ( Eq . 11 )$$

### Interaction of Ferritin With the Quantum Sensor

The iron core of ferritin represents a superparamagnetic particle that reverses the direction of its magnetisation back and forth along a single axis (referred to as its easy axis) after exponentially distributed waiting times, Δt, as defined by the average weighting time, τ_{flip} in the following autocorrelation function,

$$f ( Δ t , {τ}_{flip} ) = \{ \begin{matrix}\frac{1}{{τ}_{flip}} {e}^{- Δ t / {τ}_{flip} ,} & Δ t \geq 0 \\ 0 , & Δ t < 0\end{matrix} \qquad ( Eq . 12 )$$

where the Δt are the realisations of the times between reversal events, and τ_{flip} is the mean time between these events. The average wait times are defined by the volume V, anisotropy K, and temperature T, of the iron core, as given by the Nèel relaxation time:

$${τ}_{flip} = {τ}_{0} {e}^{KV / {k}_{b} T} \qquad ( Eq . 13 )$$

Assuming the core to be comprised of ferrihydrite, these parameters are known to be:

$$K = 3.5 \times 1 {0}^{4} J {m}^{- 3} \qquad ( Eq . 14 )$$

$${τ}_{0} = 1 {0}^{- 12} s$$

where T is the temperature and k_{b} is Boltzmann's constant. The volume of the core can be related directly to the loading factor, LF, and the density of ferrihydrite:

$$V ( LF ) = \frac{LF}{2} \times \frac{168.7 g {mol}^{- 1}}{3 \text{.8} \times {10}^{6} g {m}^{- 3}} = LF \times 3.7 \times 1 {0}^{- 2} {nm}^{3} \qquad ( Eq . 15 )$$

Given that the distribution of waiting times defines the self-correlation of the ferritin signal, the spectral distribution may be determined via its corresponding Fourier transform:

$$S ( B ; ω , γ ) = \frac{{B}^{2}}{π γ} [ \frac{{γ}^{2}}{{ω}^{2} + {γ}^{2}} ] , \qquad ( Eq . 16 )$$

where γ=1/τ_{flip} defines the width of the distribution.

The effective magnetic field B from the ferritin core can be also be written in terms of the LF. As it is the longitudinal spin dynamics of the ferritin that excite the NV transition, it is the x-z and y-z NV-Fe components of their magnetic dipole coupling tensor that are of interest:

$$\begin{matrix}{B}^{2} = {B}_{xz}^{2} + {B}_{yz}^{2} \\ = {a}^{2} {LF}^{α} \frac{( {x}^{2} + {y}^{2} ) {z}^{2}}{{( {x}^{2} + {y}^{2} + {z}^{2} )}^{5}} ,\end{matrix} \qquad ( Eq . 17 )$$

$$where a = \frac{{μ}_{0}}{4 π ℏ} {γ}_{e}^{2} \qquad ( Eq . 18 )$$

is the magnetic dipole coupling constant, γₑ is the electronic gyromagnetic ratio and the power, α, defines the number of contributing iron electron spins to the overall magnetic signal. The literature values for α vary between 0.5 and 1 depending on the magnetic model employed. For the present disclosure it has been assumed that values of α are physiologically consistent and lie within this range.

As discussed previously, for cases of practical interest the present disclosure is concerned with sensing systems comprised of many NVs and many ferritin and the response of j^{th} NV spin will thus be the result of the effective field due to all ferritin cores to which it is coupled. The full power spectrum felt by NV jis given by the sum of the individual spectra from each ferritin core (k) that comprise the signal:

$${S}_{j} ( ω ) = {\sum }_{k} \frac{{B}_{j k}^{2}}{π {γ}_{k}} [ \frac{{γ}_{k}^{2}}{{ω}^{2} + {γ}_{k}^{2}} ] , \qquad ( Eq . 19 )$$

where Bⱼₖ is the magnetic field strength felt by NV j due to ferritin core k, and γₖ is the flipping rate of ferritin core k.

The relaxation rate is then given by the convolution of this spectrum of frequencies and the filter function of the NV:

$$\begin{matrix}{Γ}_{T}^{( j )} = {\int }_{- ∞}^{∞} \frac{{Γ}_{2}}{2 ( {Γ}_{2}^{2} + {δ}^{2} )} {\sum }_{k} \frac{{B}_{jk}^{2}}{π {γ}_{k}} [ \frac{{γ}_{k}^{2}}{{ω}^{2} + {γ}_{k}^{2}} ] d ω \\ = {\sum }_{k} \frac{{B}_{jk}^{2}}{2} [ \frac{( {γ}_{k} + {Γ}_{2} )}{{( {γ}_{k} + {Γ}_{2} )}^{2} + {( 2 π D )}^{2}} ]\end{matrix} \qquad ( Eq . 20 )$$

### Quantum Sensor Geometries and Performance

As noted previously, in some embodiments it is desirable for the quantum sensor to comprise many small diamond elements, each containing one or more NV defects and these elements, referred to as nanodiamonds, may have a variety of geometries including spheres, discs, cubes and other shapes, with NV defects manufactured into them at various locations within the diamond element. These variations in sensor geometry and fabrication can affect performance of the quantum sensor as outlined in the following statistical analysis which is focused on a nanodiamond ensemble of sufficient total area to bind all ferritin in the sample while minimising measurement noise associated with NV centres not coupled to any magnetic signal arising from a ferritin.

The number of ferritin in a blood sample of volume v and ferritin mass-concentration c[F], assuming the mass of protein to be 474×10³ atomic mass units (1 amu=1.661×10⁻²⁷ kg), is:

$$\begin{matrix}N [ F ] = v \times c [ F ] / m [ F ] \\ = v \times c [ F ] \times 1.27 \times 1 {0}^{12} ferritins / mL .\end{matrix} \qquad ( Eq . 21 )$$

Particle size distributions generated by grinding, milling and crushing operations are typically represented by the Weibull distribution:

$$P ( R ; λ , k ) = \frac{k}{λ} {( \frac{R}{λ} )}^{k - 1} {e}^{- {( R / λ )}^{k}} , R > 0 \qquad ( Eq . 22 )$$

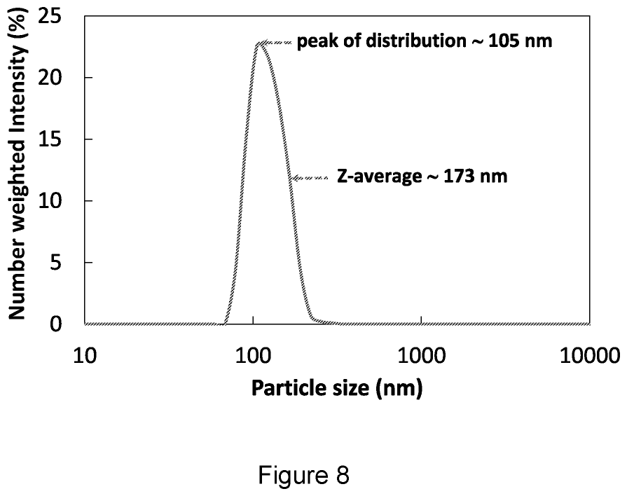

for characteristic particle size R; where the scale parameter λ, and stretch factor, k, are taken from experimental data such as that shown in FIG. 8 which is a dynamic light scattering measurement of 100 nm nanodiamond suspension. Such distributions allow for the explicit computation of the per-particle measurement statistics specified above in Eq. 10.

Given some number of nanodiamonds, N_{ND}, whose sizes are distributed as discussed above, the distribution for the total number of available sites for ferritin attachment may be given by:

$${N}_{s}^{total} = {\sum }_{k}^{{N}_{ND}} {N}_{s}^{( k )} , \qquad ( Eq . 23 )$$

where Nₛ^{(k)} is the number of available binding sites on nanodiamond k. Determining the mean of this distribution is necessary to estimate the optimal N_{ND} required to sense a given blood sample, and the spread is used to evaluate the associated coefficient of variation.

As each of the Nₛ^{(k)} are random variables, the distribution for Nₛ^{total} for a total of N_{ND} nanodimonds may be found by the (N_{ND}−1) th convolution of P_{Ns}(Nₛ) with itself:

$$P ( {N}_{s}^{total} ) = {P}_{1} ( {N}_{s}^{( 1 )} ) {\prod }_{k = 2}^{k = {N}_{ND}} \text{(*} {P}_{k} ( {N}_{s}^{( k )} ) ) \qquad ( Eq . 24 )$$

This distribution may be easily evaluated noting that between 10⁶ and 10¹⁰ nanodiamonds are required to measure loading factor for 1 mL blood sample at physiological levels, meaning that P (Nₛ^{total}) will be well approximated by a normal distribution with mean and spread

$${μ}_{Ns}^{Total} = {N}_{ND} 〈 {N}_{S} 〉 , \qquad ( Eq . 25 )$$

$${σ}_{Ns}^{Total} = {N}_{ND}^{1 / 2} \sqrt{〈 {N}_{s}^{2} 〉 - {〈 {N}_{S} 〉}^{2}} ,$$

where the first and second moments of the average number of binding sites per nanodiamond may be determined from P (R; λ, k) for some given shape distribution, as discussed below.

The required number of nanodiamonds is then given by

$${N}_{ND} = N [ F ] / 〈 {N}_{S} 〉 , \qquad ( Eq . 26 )$$

where [symbol] Nₛ [symbol] is the average number of binding sites per nanodiamond; or average number of ferritin in contact with a nanodiamond during the measurement process.

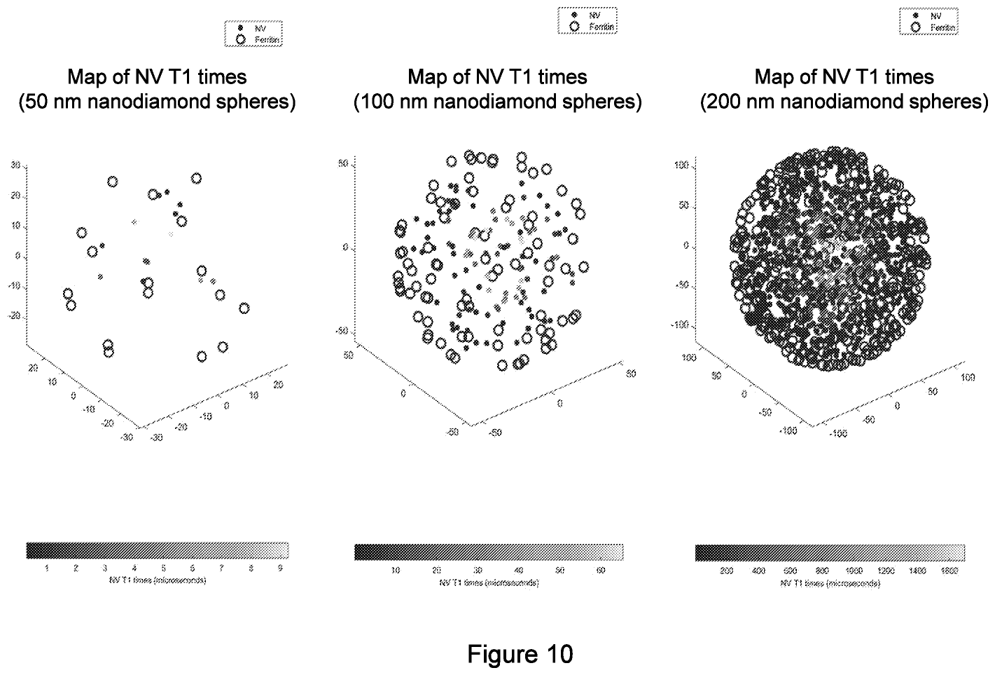

Additionally, performance metrics may be derived for various quantum sensor diamond geometries such as spherical, disc-like and single crystal nanodiamonds. FIG. 10 shows simulations of the spatial distributions of ferritins on the diamond surface and NV centres throughout the nanodiamond. The shading represents the resulting T1 relaxation times of the NV spins for 50 nm, 100 nm and 200 nm spherical nano sensing elements (nanodiamonds), with those closest to the surface of the nanodiamond sphere exhibiting the fastest relaxation rates (where relaxation rate is the inverse of relaxation (T1) time).

Owing to the large number of ferritin expected to attach to a nanodiamond surface, as well as the average isotropy of the NV-ferritin interaction, it is necessary to compute the induced relaxation rate of an arbitrary NV spin (of index j) positioned somewhere on the z axis within a nanodiamond sphere of radius Rₙ. It is useful to assume that the nanodiamond has an average ferritin surface coverage density of σₙ, and that the NVs are distributed with average bulk density nₙ.

Then, using Equations 17 and 20 above, the induced relaxation rate of NV-j may be computed explicitly via integration over all ferritin cores on the nanodiamond surface:

$${Γ}_{T}^{( j )} = \frac{( {γ}_{k} + {Γ}_{2} )}{2 [ {( {γ}_{k} + {Γ}_{2} )}^{2} + {( 2 π D )}^{2} ]} ∮ {B}_{jk}^{2} dS \qquad ( Eq . 27 )$$

$$\frac{16 π {a}^{2} {LF}^{2 α} σ ( {γ}_{k} + {Γ}_{2} )}{6 [ {( {γ}_{k} + {Γ}_{2} )}^{2} + {( 2 π D )}^{2} ]} \frac{{( {R}_{F} + {R}_{j} )}^{2} ( {z}^{2} + {( {R}_{F} + {R}_{j} )}^{2} )}{{( {( {R}_{F} + {R}_{j} )}^{2} - {z}^{2} )}^{4}}$$

where R_{F} is the effective separation distance of the ferritin above the nanodiamond surface, resulting from the radius of the ferritin and any additional species present (e.g. due to glycosylation reaction and similar effects).

Combining this target relaxation rate with the background NV relaxation rate, Γ_{BG}, and inverting as per Eq. 7 gives the contribution from NV-j, which is then summed over the nanodiamonds to evaluate its contribution to the total measurement signal. Again, owing to the large average number of NVs expected in each nanodiamond, one may approximate this by an integral over the nanodiamond volume with average NV density n. Taking advantage of the symmetry of spherical nanodiamonds, the average measurement signal within a nanodiamond size ensemble whose size distribution is described by P (Rⱼ) is given by

$$\begin{matrix}{μ}_{ND} = 〈 {\sum }_{j}^{{N}_{NV}^{( n )}} ( \frac{1}{{Γ}_{BG}^{( j )}} - \frac{1}{{Γ}_{BG}^{( j )} + {Γ}_{T}^{( j )}} ) 〉 \\ = 4 π n {\int }_{0}^{∞} {dR}_{j} P ( {R}_{j} ) {\int }_{0}^{{R}_{j}} dz {z}^{2} ( \frac{1}{{Γ}_{BG}^{( j )}} - \frac{1}{{Γ}_{BG}^{( j )} + {Γ}_{T}^{( j )}} ) .\end{matrix} \qquad ( Eq . 28 )$$

Similarly,

$${σ}_{ND}^{2} = 4 π n {\int }_{0}^{∞} {dR}_{j} P ( {R}_{j} ) {( {\int }_{0}^{{R}_{j}} dz {z}^{2} ( \frac{1}{{Γ}_{BG}^{( j )}} - \frac{1}{{Γ}_{BG}^{( j )} + {Γ}_{T}^{( j )}} ) )}^{2} - {μ}_{ND}^{2} \qquad ( Eq . 29 )$$

For a disc-like nanodiamond ensemble,

$$\begin{matrix}{Γ}_{T}^{( j )} = \frac{( {γ}_{k} + {Γ}_{2} )}{2 [ {( {γ}_{k} + {Γ}_{2} )}^{2} + {( 2 π D )}^{2} ]} \\ = \frac{4 π {a}^{2} {LF}^{2 α} σ ( {γ}_{k} + {Γ}_{2} )}{6 [ {( {γ}_{k} + {Γ}_{2} )}^{2} + {( 2 π D )}^{2} ]} \\ \frac{( {( {R}_{F} + H )}^{4} + 6 {z}^{2} + {( {R}_{F} + H )}^{2} + {z}^{4} )}{{( {R}_{F} + H - z )}^{4} {( {R}_{F} + H + z )}^{4}} ,\end{matrix} \qquad ( Eq . 30 )$$

A single crystal diamond sensor presents a somewhat complementary case to those considered above although variations in geometry are of less concern than the distribution of NV centres throughout the crystal.

In the case of a single crystal diamond, NV centres are distributed within a semi-localised plane in close proximity to the surface of the diamond crystal (less than 100 nm). In the case where ferritin has adsorbed with high density onto the diamond surface, such that the NV-NV distance is much less than the NV-ferritin distance, the ferritin contribution can be considered as continuous and the NV centres can be approximated as a 2D plane with separation H, from the layer of NV centres. In this case the induced relaxation rate of the NV ensemble may be computed explicitly as:

$$\begin{matrix}{Γ}_{T} = \frac{( \frac{1}{{τ}_{flip}} + {Γ}_{2} )}{2 [ {( \frac{1}{{τ}_{flip}} + {Γ}_{2} )}^{2} + {( 2 π D )}^{2} ]} \\ {\int }_{- ∞}^{∞} {\int }_{- ∞}^{∞} {\int }_{H}^{∞} {B}_{jk}^{2} dxdydz \\ = \frac{{a}^{2} {LF}^{2 α} π σ}{{H}^{3}} \frac{( {Γ}_{2} + \frac{1}{{τ}_{flip}} )}{( {( {Γ}_{2} + \frac{1}{{τ}_{flip}} )}^{2} + 2 π {D}^{2} )}\end{matrix} \qquad ( Eq . 31 )$$

The preceding theoretical and analytical analysis explaining the performance of NV quantum sensors (in various forms) is based on the behaviour of the NV defect/s in the quantum sensor in the presence of a molecular metal ligand or metal binding agent. Described in simple terms, the paramagnetic NV centre has a spin 1 ground and excited state which can be used to detect fluctuating, oscillating and DC magnetic fields depending on the quantum protocol employed.

In the case of ferritin, the fluctuating magnetic field from the iron core couples to the NV sensing probes causing them to depolarise. The rate of depolarisation is proportional to the loading factor of iron within ferritin. By performing measurements with and without ferritin present, the average values of Γ_{BG} and Γ_{T} can be directly determined to evaluate the average loading factor using Equation 10.

### Example—Single Crystal

To investigate the change in relaxation rate as a function of ferritin iron load, a standard set of samples were prepared which contained a narrow distribution of iron loads ranging from 10 up to 2500 Fe atoms/ferrtin. T1 relaxation measurements were carried out using a single crystal diamond hosting an ensemble of NV centres located approximately 10 nm from the diamond surface, as illustrated in FIG. 4A. A positively charged polymer coating 506 was applied prior to ferritin to aid in cleaning and adsorption.

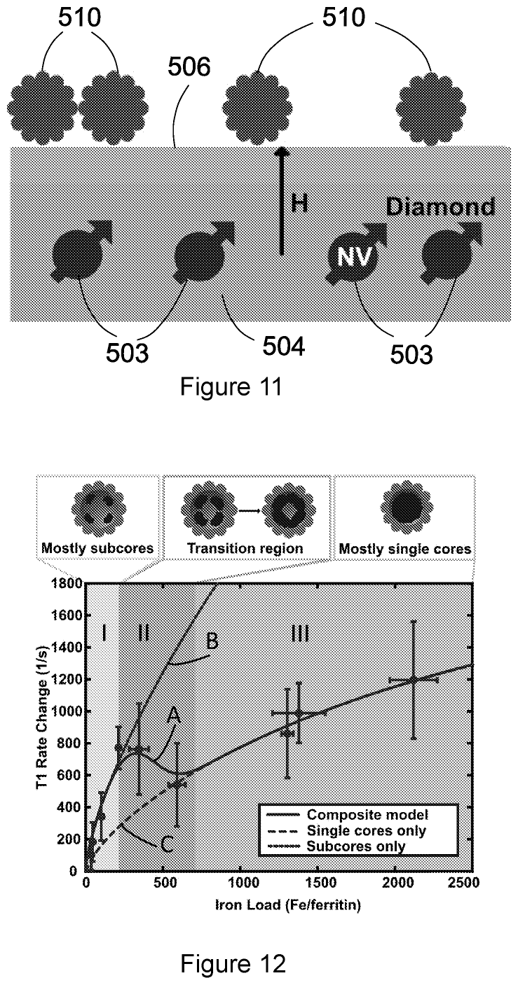

An example of the sensing configuration is shown in FIG. 11 which shows schematically the interaction between ferritin 510 and the quantum sensor 505.

The adsorbed high-density layer of ferritin starts at a distance H from the NV layer, corresponding to the situation described by Equation 31.

FIG. 12 shows the change in T₁ relaxation rate for samples containing ferritin with variable iron load after 4 hours of interaction. This data is clearly non-monotonic between iron loads of approximately 100-550 iron atoms/ferritin.

### Physical Model

Under the assumption that ferritin has a single iron core which grows with the number of Fe atoms, the change in T₁ relaxation rate can be described by Equation 31. The measured data reveals deviations from this expression which implies the single core assumption is not valid over the entire iron load range 0-4500 Fe atoms. It is understood by the inventors that for low iron loads multiple iron subcores can exist within a single ferritin. This has been proposed in previous studies utilising high-resolution electron microscopy however due to the absence of suitable technology, there has been no prior measurement of magnetic properties, as a function of iron load, to reveal this behaviour. The presence of multiple subcores will modify the strength of the fluctuating magnetic signal produced by ferrtin as it loads. To account for this morphological dependence a novel physical model is disclosed herein which includes the magnetic signal from a number of iron subcores, N_{c}, up to a particular iron load, LF_{c}, where the subcores will then fuse into a single core and grow as described by Equation 31. To account for multiple iron subcores Equation 13 must be modified to account for contributions from N_{c}, nucleation sites:

$${τ}_{subcore flip} = {τ}_{0} {e}^{KV / {N}_{c} {k}_{b} T} \qquad ( Eq . 32 )$$

Similarly, the factor α in Equation 17 is modified to be:

$${B}_{subcore}^{2} = {( \frac{1}{{N}_{C}} )}^{α} {B}^{2} \qquad ( Eq . 33 )$$

Magnetic contributions from the iron subcores will dominate at low iron loads, whereas magnetic contributions from the single cores will dominate at high iron loads. The transition between these two regimes will occur at some iron load LF_{c}. The contribution of ferritin containing subcores will have the following distribution with respect to iron load, with a transition point defined by LF_{c}:

$$F ( LF ) = exp ( - {( LF / {LF}_{c} )}^{3} . \qquad ( Eq . 34 )$$

The contribution of single cores is then given by 1−F(LF).

The new composite model can then be described by:

$${Γ}_{Composite} = F {Γ}_{Subcores} + ( 1 - F ) {Γ}_{Single} \qquad ( Eq . 35 )$$

where Γ_{single} is given by, Γ_{T}, in Equation 31. Γ_{subcores} is defined in the same way as Equation 31, however, it is evaluated using Equations 32 and 33, with the resulting composite relaxation rate given by the expression below:

$${Γ}_{composite} = \frac{{a}^{2} 4 σ {π}^{3}}{{H}^{3}} [ \frac{{LF}^{2 α} ( 1 - exp ( - {( \frac{LF}{{LF}_{c}} )}^{3} ) ) ( {Γ}_{2} + \frac{1}{{τ}_{flip}} )}{{( {Γ}_{2} + \frac{1}{{τ}_{flip}} )}^{2} + 4 {π}^{2} {D}^{2}} + \frac{\frac{{LF}^{2 α}}{{N}_{C}^{2 α}} {N}_{c} exp ( - {( \frac{LF}{{LF}_{c}} )}^{3} ) ( {Γ}_{2} + \frac{1}{{τ}_{subcore\_flip}} )}{{( {Γ}_{2} + \frac{1}{{τ}_{subcore\_flip}} )}^{2} + 4 {π}^{2} {D}^{2}} ] \qquad ( Eq . 36 )$$

The Solid line A in FIG. 12 represents data fitted using the above composite model (physical model) corresponding to Eq. 36, the tight dash line B estimates contributions from ferritin containing multiple subcores only and wide dash line C estimates ferritin containing single cores only. The discontinuity between samples containing between 212 and 591 Fe/ferritin is well reproduced by the composite model (curve A). The morphological change that occurs is represented diagrammatically above the plot of FIG. 12. For low iron loads (region I) the signal is dominated by ferritin containing multiple subcores, while for high iron loads (region III) the signal is dominated by ferritin containing a single core particle. Transition region II indicates iron loads for which there are a combination of these two modalities.

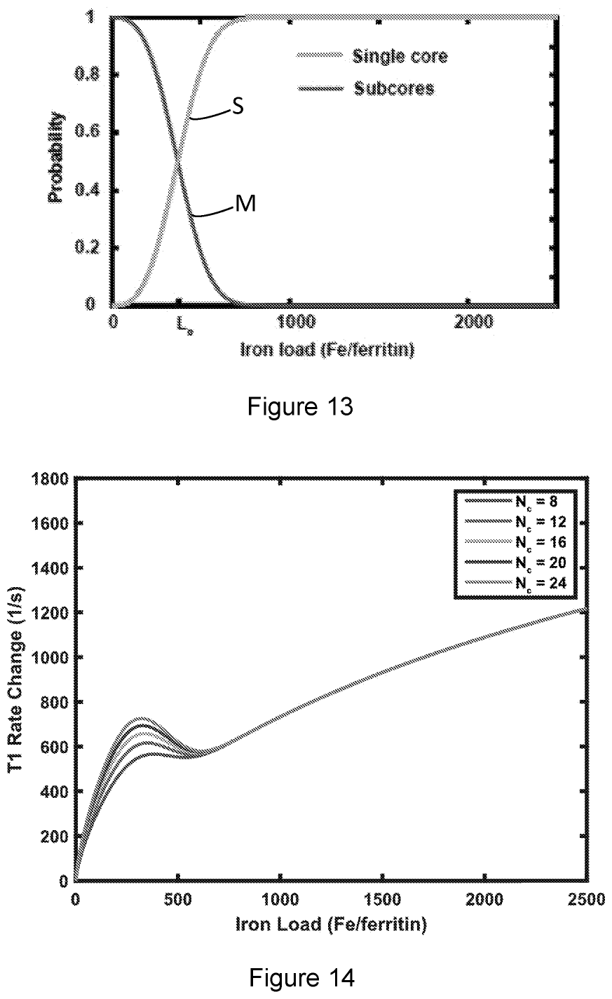

FIG. 13 is a probability distribution of ferritin containing single cores(S) and multiple cores (M) as a function of iron load. The point at which single cores dominate over multiple cores occurs at an iron load designated L_{c}. The transition between the contributions to the magnetic signal corresponding to region I and region Il occurs at approx. 400 Fe/ferritin and explains the “hump” in the data. Although the relationship is non-linear over the entire range of possible iron loads, physiological ferritin samples are traditionally less than 25% loaded (i.e. 900 Fe/ferritin on FIG. 12) thus the relationship identified according to curve A can be utilised to infer iron load in ferritin serum samples.

Approaches are provided in this disclosure for determining a mathematical model representative of curve A for determining iron load using T₁ measurements according to the embodiments disclosed herein. Both non-physical and physical approaches are exemplified, however one skilled in the art would appreciate that different approaches may be adopted. These models, in particular the physical model, may be formulated based on a basic explanation of the relationship between iron load in ferritin and T₁ rate.

Ferritin is a hollow globular protein consisting of 24 subunits of two types. Its make-up is such that the iron core is in general thought to nucleate at sites on one of the subunit types, resulting in one to twenty-four nucleation sites per ferritin molecule. This morphological property results in a non-linear relationship between the measured T₁ relaxation rate from the NV defects in the quantum sensor and the iron load of the ferritin molecules. It is envisaged that this non-monotonic behaviour will be present regardless of ferritin origin, whether that be from different tissues or species.

At the two extremes of high (>1000 Fe/ferritin) and low (<400 Fe/ferritin) iron load, the relationship between T₁ relaxation rate and iron load is expected to follow a monotonically increasing power law with power between 0 and 1.

Relying on a theoretical model which describes how the magnetic properties of the iron core evolve as a function of nucleation sites, the non-linear behaviour is determined to be dependent on the number of nucleation sites. This is presented in FIG. 14 which represents the relationship between the change in T₁ relaxation rate and iron load for a given number of nucleation sites Nc within each ferritin.

By taking into account the number of nucleation sites and volume constraints of ferritin it is possible, in the absence of experimental data to build a mathematical model based on physical assumptions (physical model) which describes how the magnetic signal from the iron core evolves as a function of iron atoms within ferritin. The physically derived model (curve A in FIG. 12), had excellent agreement with the measured T₁ signal as a function of iron load. This relationship can be used to provide an accurate assessment of the iron load for a particular sample origin or species.

### Non-Physical Model

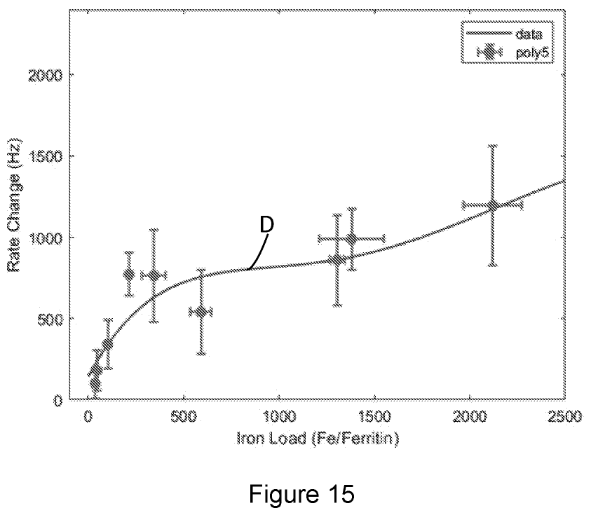

Using a non-linear least squares fitting algorithm it is possible to fit the data without first developing a physically motivated model. Taking this approach, one can generate a fit using a range of equations given the right parameter bounds and x-domain. The curve D of FIG. 15 is just one example, using a 5th order polynomial which fits the data across the region of interest. Although curve D does not account well for the declining portion of the “hump” in the data in FIG. 12, it still provides for meaningful determination and qualitative assessment of iron load such as “low”, “medium” or “high” to be inferred. Similar results may be achieved using a myriad of other known and widely used curve fitting approaches as would be understood by one skilled in the art.

### Applications

The strong agreement between the theoretical modelling and experimental results confirms the validity of the techniques disclosed herein, and demonstrates they can possess the requisite sensitivity and repeatability to be able to measure iron load in ferritin from physiological blood samples. There are numerous applications for the sensing methods and systems (techniques) disclosed herein, some of which are discussed below.

Management of iron overload is generally by venesection, with the protein content of serum ferritin being used to monitor the condition and determine the frequency of treatment (perhaps once a week for 12 months or more). A more accurate, direct measurement of iron load would transform management of hemochromatosis, avoiding venesection (and causing iron deficiency) in patients with high serum ferritin levels but low iron load.

Additionally, ferritin iron load may have wide influence on neuronal health, from stroke recovery to stem cell transplantation success and may be a key modulator of Alzheimer's disease. A direct measure of ferritin-iron load according to embodiments of the present disclosure opens up the possibility of exploring the role that in-vivo ferritin-iron plays in neuronal health and regeneration.

Techniques disclosed herein may complement existing clinical blood tests, including immunochemical tests for ferritin quantification. These techniques offer the potential for advancement in accurate diagnoses and treatment planning since the currently unmet clinical need for diagnosing both positive and negative iron status affects disease susceptibility and is critical for instructing a patient on treatment and management. This is particularly relevant for patients at risk of developing or currently managing hemochromatosis, and for identifying iron deficiency when concurrent with inflammation. Furthermore, determination of ferritin bound iron could target individuals for genetic testing to efficiently facilitate diagnosis of primary hereditary hemochromatosis, and those with non-specific high-ferritin (apoferritin) can be directed to appropriate disease-specific treatment.

For those patients correctly diagnosed with hemochromatosis, accurate determination of iron loading of ferritin would be useful for the de-ironing (venesection) stage of hemochromatosis treatment. Early diagnosis and prompt initiation of treatment (e.g. by venesection) significantly improves prognosis. Left untreated, the long term effects of hemochromatosis can include liver cancer, cardiomyopathy, diabetes and cirrhosis. Current treatment relies on the level of serum ferritin rather than level of iron stored however these levels can become elevated due to other reasons such as inflammation and alcohol consumption. Ideally, the techniques disclosed herein would provide a method for planning venesection so that it occurs only when iron concentration is high.

The techniques disclosed herein set a foundation for rapid, accurate testing of iron load using an inexpensive to manufacture quantum sensor. Unlike FerriScan and other magnetic resonance imaging techniques, the infrastructure for performing the detection is inexpensive with low running costs. The system could be made portable, and even battery operated making it a viable instrument for mobile health screening and deployment in remote locations.

The electron spin relaxation (ESR) based techniques disclosed herein offer several advantages over both conventional and existing quantum sensing techniques. Measurements of the NV relaxation due to the presence of ferritin in general does not require microwave control, and thus requires no coherent control of the sample. With relaxation times much longer than dephasing times, T1-based protocols can be significantly more sensitive to ESR detection. Finally, even in the ensemble case demonstrated here, the NV spin relaxation is dominated by local interactions with the environment which affords an effective spatial resolution of a few nanometres.

Unlike MRI, the techniques of the present disclosure do not measure relaxation coherence of H₂O molecules in the presence of high iron/ferritin concentration. Rather, a specifically engineered quantum sensor having a manufactured defect, ideally an NV defect, is utilised. When exposed to small concentrations of ferritin of different iron load, the spin relaxation time of the NV defect changes and this can be detected optically. This approach offers sensitivity improvements over MRI and is significantly more cost and time effective as a routine assay. Furthermore, the patient is not required to be present for the analysis phase of the test. Rather, a blood sample can be taken relatively quickly and the patient can carry on with other activities while awaiting the results of the analysis.

While applications of the present disclosure have been presented in the context of detection of iron load in ferritin, it is to be understood that many and varied other applications exist in nanofabrication, chemistry and biochemistry. For example, ferritin can be used as a cage to store other biomolecules such as magnetite, as a containment vessel for microscopic reactions such as synthesis of iron oxide nanoparticles, or as a delivery vehicle for drugs. Therefore, the techniques disclosed herein which utilize quantum sensing to detect, by optical methods, a change in spin state attributable to the property (such as loading factor) of a molecular metal ligand or metal binding agent have wide and varied potential.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that various modifications, additions and/or alterations may be made to the parts previously described without departing from the ambit of the present invention as defined in the claims appended hereto.

Future patent applications may be filed on the basis of the present application. It is to be understood that the following claims are provided by way of example only, and are not intended to limit the scope of what may be claimed in any such future application. Features may be added to or omitted from the claims at a later date so as to further define or re-define the invention or inventions.

1. A method for determining one or more properties of a molecular metal ligand in a sample, comprising the steps of:
a. providing a quantum sensor;
b. exposing the quantum sensor to the sample;
c. applying an illumination signal to the quantum sensor for a first predetermined duration; and
d. detecting a photoluminescence intensity emitted from the quantum sensor;
wherein a characteristic of the detected photoluminescence intensity is indicative of one of the properties of the molecular metal ligand in the sample.

2. The method according to claim 1, wherein the applied illumination signal has a wavelength in a range of about 415 nm to about 630 nm, preferably about 480 nm to about 560 nm and more preferably about 532 nm.

3. The method according to claim 1 or claim 2, wherein the applied illumination signal comprises a pulsed signal having an excitation phase of about 200 ns to about 100 μs, preferably about 2 μs to about 20 μs and more preferably about 5 μs, followed by an interaction time, and optionally, wherein the interaction time between excitation phases is from about 12 ns to about 10 ms.

4. The method according to any one of the preceding claims, wherein the detected photoluminescence intensity is measured during an excitation phase of the illumination signal applied to the quantum sensor.

5. The method according to any one of the preceding claims, comprising the step of detecting the photoluminescence intensity at one or more wavelengths in a range of about 620 nm to about 850 nm, preferably between about 637 nm to about 800 nm.

6. The method according to any one of the preceding claims, comprising the step of measuring rate of decay of the detected photoluminescence intensity, wherein the rate of decay (1/T₁) indicates a property of the sample corresponding to loading factor of the molecular metal ligand.

7. The method according to any one of the preceding claims, comprising the step of comparing the measured rate of decay of the detected photoluminescence intensity with a background rate of decay measured from the quantum sensor when the illumination signal is applied in the absence of the sample.

8. The method according to any one of the preceding claims, wherein the molecular metal-ligand is selected from a group comprising metalloproteins, metal-chelating agents and other metal-binding agents.

9. The method according to claim 8, wherein the metalloprotein is ferritin.

10. The method according to claim 9, wherein the property is loading factor which is indicative of iron bound to ferritin in the sample.

11. The method according to claim 8, wherein the property is loading factor which is indicative of an extent of loading of a metal or metalloenzyme within the molecular metal ligand and optionally, wherein the metal is selected from a group comprising vanadium, manganese, iron, cobalt, nickel, copper, gadolinium, and cadmium.

12. The method according to any one of the preceding claims, wherein the quantum sensor comprises an addressable spin defect in a semiconductor material.

13. The method according to claim 12, wherein the semiconductor is a diamond preferably produced via chemical vapour deposition (CVD) or high-pressure-high-temperature (HPHT) processes.

14. The method according to claim 12 or claim 13, wherein the spin defect is a nitrogen-vacancy (NV) defect that has been engineered in the semiconductor material.

15. The method according to any one of claims 12 to 14, wherein the quantum sensor comprises one or both of:
(a) NV defects at a density of from about 0.001 to about 500 parts-per-million relative to the semiconductor site density, preferably about 1 part-per-million;
(b) NV defects located less than about 100 nm, preferably less than about 20 nm, more preferably less than about 10 nm from the semiconductor material surface.

16. The method according to any one of the preceding claims, wherein the quantum sensor comprises a plurality of quantum sensor elements and wherein exposing the quantum sensor elements to the sample comprises providing the quantum sensor elements in suspension within a fluid comprising the sample.

17. The method according to claim 16, wherein the plurality of quantum sensor elements have a geometry selected from the group comprising randomly shaped chunks, spherical, disc-like and single crystal elements.

18. The method according to claim 16 or claim 17, wherein the plurality of quantum sensor elements have a diameter of between about 20 nm and about 500 nm, preferably between about 50 nm and about 200 nm.

19. The method according to any one of the preceding claims, wherein the sample is a biological sample, preferably a biological fluid sample.

20. The method according to claim 19, wherein the biological fluid sample is selected from a group comprising: blood, blood serum, blood plasma, cerebrospinal fluid, urine, saliva, pericardial fluid, pleural fluid, synovial fluid, amniotic fluid, seminal fluid, sweat and tears.

21. The method according to claim 19 or claim 20, comprising the step of preparing the biological fluid sample by performing one or more of centrifuging the fluid, heating the fluid, passing the fluid through a liquid chromatograph or selective membrane, modifying pH and performing immuno or affinity capture to simplify the fluid for determining properties of one or more target species within the biological fluid sample.

22. The method according to any one of the preceding claims, wherein the photoluminescence intensity is detected using a CCD (charge-coupled device) or a complementary metal oxide semiconductor (sCMOS) and optionally, wherein the detected photoluminescence intensity conveys uniformity of the biological sample with respect to the quantum sensor.

23. The method according to any one of claims 1 to 21, wherein the photoluminescence intensity is detected using a photodiode.

24. Use of the method according to any one of the preceding claims in an apparatus, system or protocol for diagnosing one or more of iron deficiency, iron deficiency anaemia, iron overload and inflammation.

25. Use of the method according to any one of the preceding claims in a method, system or apparatus guiding management and/or treatment of one or more of iron deficiency, iron deficiency anaemia, haemochromatosis, iron overload and clinically diagnosed inflammation.

26. A system for detecting one or more properties of a molecular metal ligand in a sample, the system comprising:
(a) a quantum sensor configured to be exposed to the sample;
(b) an illumination source configured to apply an illumination signal to the quantum sensor;
(c) a detector configured to detect a photoluminescence intensity emitted from the quantum sensor; and
(d) a controller configured to control operation of the illumination source to deliver pulsed illumination;
wherein a characteristic of the detected photoluminescence is indicative of one of the properties of the molecular metal ligand in the sample.

27. The system according to claim 26, wherein the controller controls operation of the illumination source to deliver pulses of illumination having a duration of about 200 ns to about 100 μs, preferably about 2 μs to about 20 μs and more and more preferably about 5 μs.

28. The system according to claim 26 or claim 27, wherein the controller controls operation of the illumination source to space pulses of illumination by a time duration from about 12 ns to about 10 ms.

29. The system according to claim 28 wherein the controller controls operation of the illumination source to divert a beam path away from the quantum sensor between pulses of illumination.

30. The system according to claim 28, wherein the controller controls operation of the illumination source to extinguish illumination between pulses of illumination.

31. The system according to any one of claims 26 to 30, wherein the controller controls operation of the detector to detect photoluminescence intensity during an excitation phase of the pulsed illumination.

32. The system according to any one of claims 26 to 31, wherein the controller includes or is in operable communication with a processor configured to determine rate of decay (1/T₁) of the detected photoluminescence intensity, wherein the determined rate of decay indicates a property of the sample corresponding to loading factor of the molecular metal ligand.

33. The system according to claim 32, wherein the processor is configured to compare the measured rate of decay with a background rate of decay measured from the quantum sensor when the illumination signal is applied in the absence of the sample.

34. The system according to claim 32 or claim 33, wherein the processor is configured to determine automatically, from a mathematical model, calibration curve or lookup table stored in memory associated with the processor, loading factor for the molecular metal ligand, wherein the calibration curve or lookup table associates values of rate of decay with values for loading factor.

35. The system according to claim 34, wherein the processor is configured to:
receive or determine a value representing concentration of the molecular metal ligand in the sample; and
determine automatically a concentration of a target species within the sample by multiplying a value representing the loading factor with the value representing the concentration of the molecular metal ligand.

36. The system according to any one of claims 26 to 35, wherein the illumination source is configured to emit illumination at one or more wavelengths in a range of about 415 nm to about 630 nm, preferably in a range of about 480 nm to about 560 nm, and more preferably about 532 nm.

37. The system according to any one of claims 26 to 36, wherein the quantum sensor comprises an addressable spin defect in a semiconductor material.

38. The system according to claim 37, wherein the semiconductor material is a diamond preferably produced via chemical vapour deposition (CVD) or high-pressure-high-temperature (HPHT) processes.

39. The system according to claim 37 or claim 38, wherein the spin defect is a nitrogen-vacancy (NV) defect that has been engineered in the semiconductor material.

40. The system according to any one of claims 26 to 39, wherein the quantum sensor comprises a plurality of quantum sensor elements, and wherein the system comprises a vessel for providing the quantum sensor elements in suspension within a fluid comprising the sample.

41. The system according to claim 40, wherein the plurality of quantum sensor elements have a geometry selected from the group comprising randomly shaped chunks, spherical, disc-like and single crystal.

42. The system according to claim 40 or 41, wherein the plurality of quantum sensor elements have a diameter of between about 20 nm and about 500 nm, preferably between about 50 nm and about 200 nm

43. The system according to any one of claims 26 to 42, wherein the sample is a biological sample, preferably a biological fluid sample.

44. The system according to any one of claims 26 to 43, wherein the detector comprises one or more of a charge-coupled device (CCD), a complementary metal-oxide-semiconductor (CMOS) sensor, and a photodiode.

45. The system according to any one of claims 26 to 44, wherein the quantum sensor, the illumination source and the detector are contained in an optically sealed housing that prevents incursion of light from outside the housing while in use.

46. The system of claim 34 or any one of claims 35 to 45 when appended to claim 34, wherein the mathematical model represents a plurality of physical properties of the molecular metal ligand.

47. The system of claim 34 or any one of claims 35 to 45 when appended to claim 34, wherein the mathematical model approximates a relationship between values of rate of decay and loading factor in experimental data obtained from one or more samples of the molecular metal ligand.
